(19) United States
(12) Patent Application Publication
(10) Pub. No.: US 2023/0393479 A1
(43) Pub. Date: Dec. 7, 2023

(54) RESIST UNDERLAYER FILM-FORMING COMPOSITION HAVING BENZYLIDENECYANOACETATE GROUP

(71) Applicant: NISSAN CHEMICAL CORPORATION, Tokyo (JP)

(72) Inventors: Shun KUBODERA, Toyama-shi (JP); Tokio NISHITA, Toyama-shi (JP); Yuki ENDO, Toyama-shi (JP); Takahiro KISHIOKA, Toyama-shi (JP)

(73) Assignee: NISSAN CHEMICAL CORPORATION, Tokyo (JP)

(21) Appl. No.: 18/278,652

(22) PCT Filed: Mar. 2, 2022
(86) PCT No.: PCT/JP2022/008706

§ 371 Date: Aug. 24, 2023

(30) Foreign Application Priority Data
Mar. 3, 2021 (JP) 2021-033326

(51) Int. Cl. G03F 7/11; C07D 251/34; C08F 20/36; C09D 133/14; H01L 21/288; H01L 21/027

(52) U.S. Cl. CPC G03F 7/11; C07D 251/34; C08F 20/36; C09D 133/14; H01L 21/288; H01L 21/0272

(57) ABSTRACT

A resist underlayer film-forming composition contains: (A) a compound having a partial structure represented by Formula (1). In Formula (1), R₁ and R₂ each denote a hydrogen atom, an alkyl group having 1-10 carbon atoms or an aryl group having 6-40 carbon atoms, X denotes an alkyl group having 1-10 carbon atoms, a hydroxyl group, an alkoxy group having 1-10 carbon atoms, an alkoxycarbonyl group having 1-10 carbon atoms, a halogen atom, a cyano group, a nitro group or a combination of these, Y denotes a direct bond, an ether bond, a thioether bond or an ester bond, n is an integer between 0 and 4, and * denotes a site of bonding to a residue of compound (A)); and a solvent.

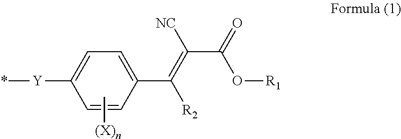

### TECHNICAL FIELD

The present invention relates to a resist underlayer film-forming composition, a resist underlayer film obtained from the resist underlayer film-forming composition, a patterned substrate production method and a semiconductor device manufacturing method using the resist underlayer film-forming composition, and a compound having a benzylidenecyanoacetate group and a method for producing the same.

### BACKGROUND ART

In semiconductor manufacturing, a lithography process is widely known, in which a resist underlayer film is provided between a substrate and a resist film that is formed thereon, and a resist pattern is formed with a desired shape. After the resist pattern is formed, the resist underlayer film is removed and the substrate is processed. These steps are mainly performed by dry etching. Dry etching is also used in the step of removing the resist pattern and the underlying resist underlayer film that are no longer necessary after the substrate processing. Wet etching with a chemical is sometimes employed in order to simplify the process steps and to reduce the damage to the workpiece substrate.

Patent Literature 1 discloses an anti-reflective coating composition with improved spin bowl compatibility.

### CITATION LIST

### Patent Literature

Patent Literature 1: JP 2004-533637 A

### SUMMARY OF INVENTION

### Technical Problem

Resist underlayer films are required to have high resistance to resist solvents so as not to separate or not to be damaged upon contact with any resist solvent in order to ensure that the resist applied on the resist underlayer film will form a desired resist pattern by exposure to a radiation (for example, ArF excimer laser beam, KrF excimer laser beam, or i-radiation) followed by development. Furthermore, resist underlayer films are also required to have high resistance to resist developers so as not to separate or not to be damaged upon contact with any resist developer (an aqueous alkali solution) mainly used in the resist development step. Furthermore, resist underlayer films are required to have anti-reflection performance, specifically, are required to eliminate or reduce the reflection of a lithography radiation from the underlying substrate and thereby to minimize or prevent deterioration of a resist pattern due to standing waves, thereby ensuring that a desired resist pattern will be obtained. Moreover, resist underlayer films are required to have a high etching speed (a high etching rate) so that the resist underlayer film can be removed quickly by dry etching and the underlying substrate will not be damaged during the dry etching removal of the resist underlayer film. When, in particular, resist underlayer films are removed by wet etching with a chemical, the resist underlayer films are required to exhibit sufficient solubility with respect to the wet etching chemical and to be easily removed from the substrate.

Meanwhile, as wet etching chemicals for removing resists and resist underlayer films, organic solvents are used for the purpose of reducing damage to the workpiece substrates. Moreover, basic organic solvents are used to enhance the removal of resists and resist underlayer films. Unfortunately, the conventional resist underlayer films have a limited performance in exhibiting such properties as being removable by, preferably soluble in, wet etching chemicals exclusively, while showing good resistance to resist solvents that are mainly organic solvents and to resist developers that are aqueous alkali solutions. An object of the present invention is to solve the problems discussed above.

### Solution to Problem

The present invention embraces the following.

[1]

A resist underlayer film-forming composition comprising a solvent and a compound (A) containing a partial structure represented by Formula (1) below,

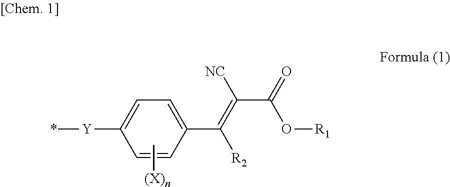

wherein R₁ and R₂ each denote a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; Y denotes a direct bond, an ether bond, a thioether bond, or an ester bond; n denotes an integer of 0 to 4; and * denotes a bond to a remaining moiety of the compound (A)).

[2]

The resist underlayer film-forming composition according to [1], wherein the compound (A) is represented by Formula (2):

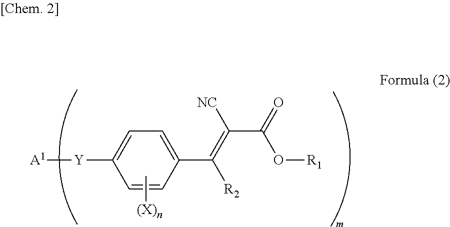

wherein A¹ denotes an m-valent organic group; m denotes an integer of 1 to 10; R₁ and R₂ each denote a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; Y denotes a direct bond, an ether bond, a thioether bond, or an ester bond; and n denotes an integer of 0 to 4.

[3]

The resist underlayer film-forming composition according to [2], wherein A¹ comprises a heterocyclic ring.

[4]

The resist underlayer film-forming composition according to [3], wherein the heterocyclic ring is triazinetrione.

[5]

The resist underlayer film-forming composition according to any one of [2] to [4], wherein the compound (A) is a reaction product of:
a compound (a) having in quantity of epoxy group(s);
a compound (b) represented by Formula (b) below:

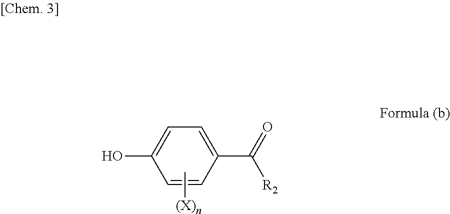

wherein R₂ denotes a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; and n denotes an integer of 0 to 4); and
a compound (c) represented by Formula (c) below:

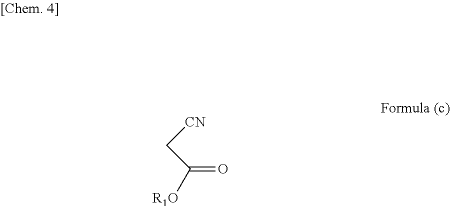

wherein R₁ denotes a hydrogen atom or an optionally substituted C1-C10 alkyl group.

[6]

The resist underlayer film-forming composition according to any one of [1] to [5], further comprising at least one member selected from the group consisting of crosslinking agents, acids, and acid generators.

[7]

The resist underlayer film-forming composition according to any one of [1] to [6], which is for application to a substrate having copper on a surface.

[8]

A resist underlayer film obtained by removing the solvent from a coating film comprising the resist underlayer film-forming composition according to any one of [1] to [7].

[9]

A resist underlayer film comprising a dried or concentrated resist underlayer film-forming composition according to any one of [1] to [7].

[10]

The resist underlayer film according to [8] or [9], which is formed on a substrate having copper on a surface.

[11]

A substrate comprising a copper seed layer on a surface, and the resist underlayer film according to [8] or [9] formed on the copper seed layer.

[12]

A method for producing a patterned substrate, comprising the steps of:
applying the resist underlayer film-forming composition according to any one of [1] to [7] onto a substrate having copper on a surface, and performing baking to form a resist underlayer film;
applying a resist onto the resist underlayer film and performing baking to form a resist film;
exposing the semiconductor substrate coated with the resist underlayer film and the resist; and
developing the exposed resist film, and performing patterning.

[13]

A method for manufacturing a semiconductor device, comprising the steps of:
forming. on a substrate having copper on a surface, a resist underlayer film from the resist underlayer film-forming composition according to any one of [1] to [7];
forming a resist film on the resist underlayer film;
forming a resist pattern by applying a light or electron beam to the resist film followed by development, and removing the resist underlayer film exposed between the resist pattern;
plating a region exposed between the resist pattern with copper; and
removing the resist pattern and the resist underlayer film beneath the resist pattern.

[14]

The method according to [13], wherein at least one of the steps of removing the resist underlayer film is performed by wet treatment.

[15]

A compound (A) represented by Formula (2) below:

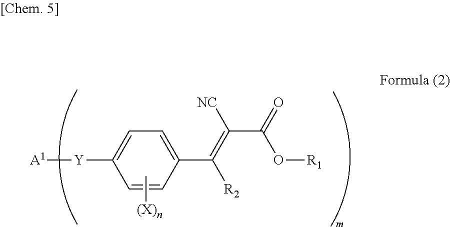

wherein A¹ denotes an m-valent organic group; m denotes an integer of 1 to 10; R₁ and R₂ each denote a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; Y denotes a direct bond, an ether bond, a thioether bond, or an ester bond; and n denotes an integer of 0 to 4.

### Advantageous Effects of Invention

The resist underlayer film provided by the present invention can exhibit such properties as being removable by, preferably soluble in, wet etching chemicals exclusively, while showing good resistance to resist solvents that are mainly organic solvents as well as to resist developers that are aqueous alkali solutions.

### DESCRIPTION OF EMBODIMENTS

<Resist Underlayer Film-Forming Composition>

A resist underlayer film-forming composition of the present invention contains a solvent and a compound (A) containing a partial structure represented by Formula (1) below,

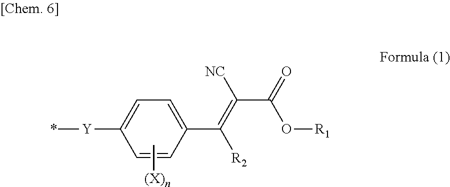

wherein R₁ and R₂ each denote a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; Y denotes a direct bond, an ether bond, a thioether bond, or an ester bond; n denotes an integer of 0 to 4; and * denotes a bond to a remaining moiety of the compound (A)).

Examples of the C1-C10 alkyl groups include methyl group, ethyl group, n-propyl group, i-propyl group, cyclopropyl group, n-butyl group, i-butyl group, s-butyl group, t-butyl group, cyclobutyl group, 1-methyl-cyclopropyl group, 2-methyl-cyclopropyl group, n-pentyl group, 1-methyl-n-butyl group, 2-methyl-n-butyl group, 3-methyl-n-butyl group, 1,1-dimethyl-n-propyl group, 1,2-dimethyl-n-propyl group, 2,2-dimethyl-n-propyl group, 1-ethyl-n-propyl group, cyclopentyl group, 1-methyl-cyclobutyl group, 2-methyl-cyclobutyl group, 3-methyl-cyclobutyl group, 1,2-dimethyl-cyclopropyl group, 2,3-dimethyl-cyclopropyl group, 1-ethyl-cyclopropyl group, 2-ethyl-cyclopropyl group, n-hexyl group, 1-methyl-n-pentyl group, 2-methyl-n-pentyl group, 3-methyl-n-pentyl group, 4-methyl-n-pentyl group, 1,1-dimethyl-n-butyl group, 1,2-dimethyl-n-butyl group, 1,3-dimethyl-n-butyl group, 2,2-dimethyl-n-butyl group, 2,3-dimethyl-n-butyl group, 3,3-dimethyl-n-butyl group, 1-ethyl-n-butyl group, 2-ethyl-n-butyl group, 1,1,2-trimethyl-n-propyl group, 1,2,2-trimethyl-n-propyl group, 1-ethyl-1-methyl-n-propyl group, 1-ethyl-2-methyl-n-propyl group, cyclohexyl group, 1-methyl-cyclopentyl group, 2-methyl-cyclopentyl group, 3-methyl-cyclopentyl group, 1-ethyl-cyclobutyl group, 2-ethyl-cyclobutyl group, 3-ethyl-cyclobutyl group, 1,2-dimethyl-cyclobutyl group, 1,3-dimethyl-cyclobutyl group, 2,2-dimethyl-cyclobutyl group, 2,3-dimethyl-cyclobutyl group, 2,4-dimethyl-cyclobutyl group, 3,3-dimethyl-cyclobutyl group, 1-n-propyl-cyclopropyl group, 2-n-propyl-cyclopropyl group, 1-i-propyl-cyclopropyl group, 2-i-propyl-cyclopropyl group, 1,2,2-trimethyl-cyclopropyl group, 1,2,3-trimethyl-cyclopropyl group, 2,2,3-trimethyl-cyclopropyl group, 1-ethyl-2-methyl-cyclopropyl group, 2-ethyl-1-methyl-cyclopropyl group, 2-ethyl-2-methyl-cyclopropyl group, 2-ethyl-3-methyl-cyclopropyl group, and decyl group.

Examples of the C6-C40 aryl groups include phenyl group, o-methylphenyl group, m-methylphenyl group, p-methylphenyl group, o-chlorophenyl group, m-chlorophenyl group, p-chlorophenyl group, o-fluorophenyl group, p-fluorophenyl group, o-methoxyphenyl group, p-methoxyphenyl group, p-nitrophenyl group, p-cyanophenyl group, a-naphthyl group. P-naphthyl group, o-biphenylyl group, m-biphenylyl group, p-biphenylyl group, I-anthryl group, 2-anthryl group, 9-anthryl group, 1-phenanthryl group, 2-phenanthryl group, 3-phenanthryl group, 4-phenanthryl group, and 9-phenanthryl group.

Examples of the C1-C10 alkoxy groups include groups formed by bonding of an oxygen atom to the alkyl groups enumerated above, such as, for example, methoxy group, ethoxy group, n-propoxy group, i-propoxy group, n-butoxy group, i-butoxy group, s-butoxy group, t-butoxy group, n-pentoxy group, 1-methyl-n-butoxy group, 2-methyl-n-butoxy group, 3-methyl-n-butoxy group, 1,1-dimethyl-n-propoxy group, 1,2-dimethyl-n-propoxy group, 2,2-dimethyl-n-propoxy group, 1-ethyl-n-propoxy group, n-hexyloxy group, 1-methyl-n-pentyloxy group, 2-methyl-n-pentyloxy group, 3-methyl-n-pentyloxy group, 4-methyl-n-pentyloxy group, 1,1-dimethyl-n-butoxy group, 1,2-dimethyl-n-butoxy group, 1,3-dimethyl-n-butoxy group, 2,2-dimethyl-n-butoxy group, 2,3-dimethyl-n-butoxy group, 3,3-dimethyl-n-butoxy group, 1-ethyl-n-butoxy group, 2-ethyl-n-butoxy group, 1,1,2-trimethyl-n-propoxy group, 1,2,2-trimethyl-n-propoxy group, 1-ethyl-1-methyl-n-propoxy group, 1-ethyl-2-methyl-n-propoxy group, n-heptyloxy group, n-octyloxy group, n-nonyloxy group, and n-decanyloxy group.

Examples of the C1-C10 alkoxycarbonyl groups include groups formed by bonding of an oxygen atom and a carbonyl group to the alkyl groups enumerated above, such as, for example, methoxycarbonyl group, ethoxycarbonyl group, propoxycarbonyl group, and butoxycarbonyl group.

Examples of the halogen atoms include fluorine atom, chlorine atom, bromine atom, and iodine atom.

The compound (A) may be represented by Formula (2):

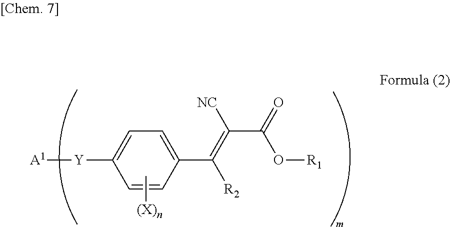

wherein A¹ denotes an m-valent organic group; m denotes an integer of 1 to 10; R₁ and R₂ each denote a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; Y denotes a direct bond, an ether bond, a thioether bond, or an ester bond; and n denotes an integer of 0 to 4.

The m-valent organic group A¹ is not limited as long as the advantageous effects of the subject application are achieved, and may be an organic group that includes a heterocyclic ring, an aromatic ring, or a hydrocarbon group optionally interrupted by an oxygen atom.

A¹ may include a heterocyclic ring.

The meaning of heterocyclic compounds in the present invention is encompassed in the definition of the term commonly used in organic chemistry. The heterocyclic compounds are not particularly limited. Examples thereof include furan, thiophene, pyrrole, imidazole, pyran, pyridine, pyrimidine, pyrazine, pyrrolidine, piperidine, piperazine, morpholine, quinuclidine, indole, purine, quinoline, isoquinoline, chromene, thianthrene, phenothiazine, phenoxazine, xanthene, acridine, phenazine, carbazole, hydantoin, triazine, and cyanuric acid. The heterocyclic ring may be triazinetrione.

Examples of the epoxy group-containing compounds for producing the compound (A) of the subject application, specifically, the compounds (a) having m quantity of epoxy group(s) (m is the same as defined above) include compounds illustrated below that include compounds represented by Formulas (B-1) to (B-18).

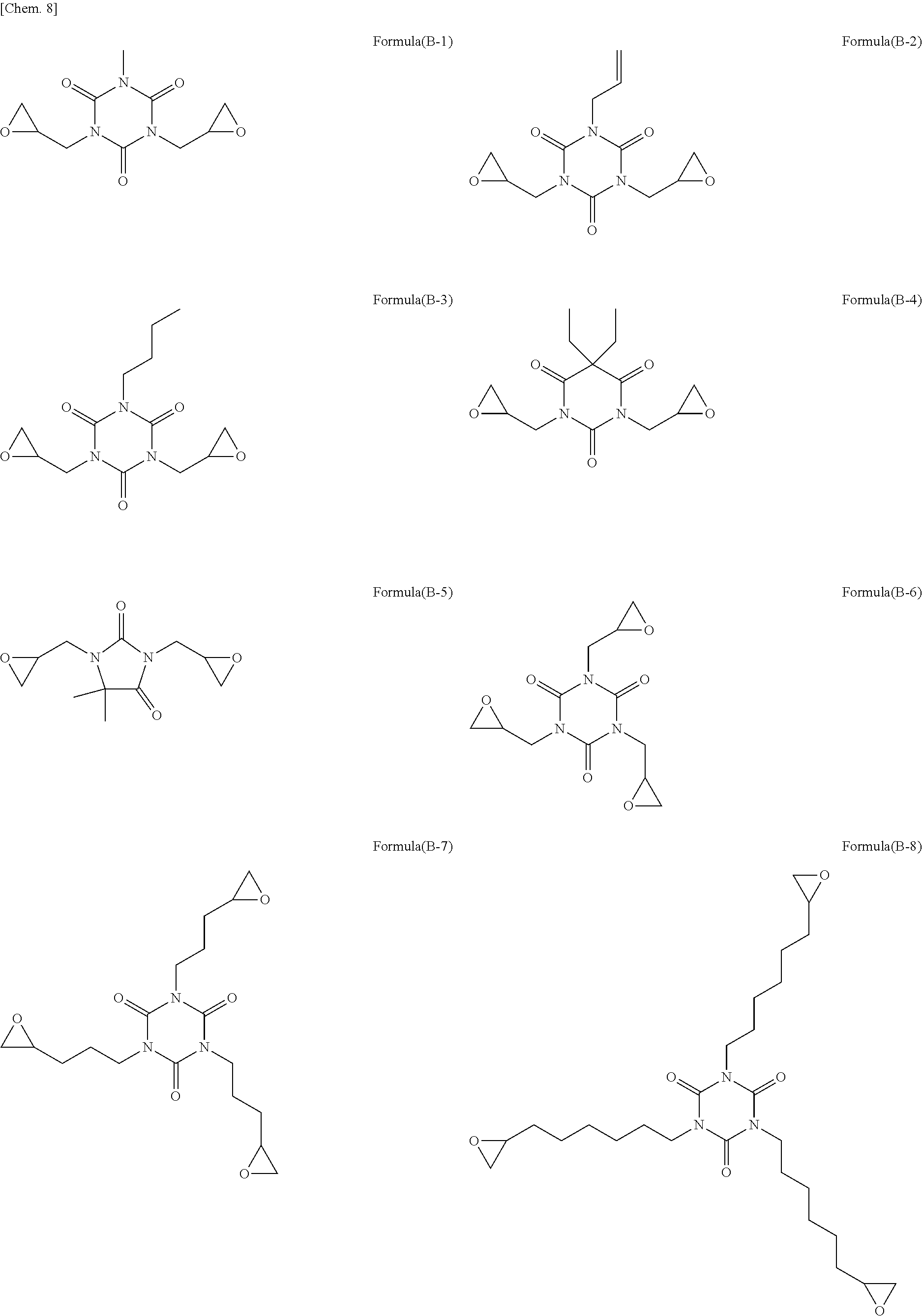

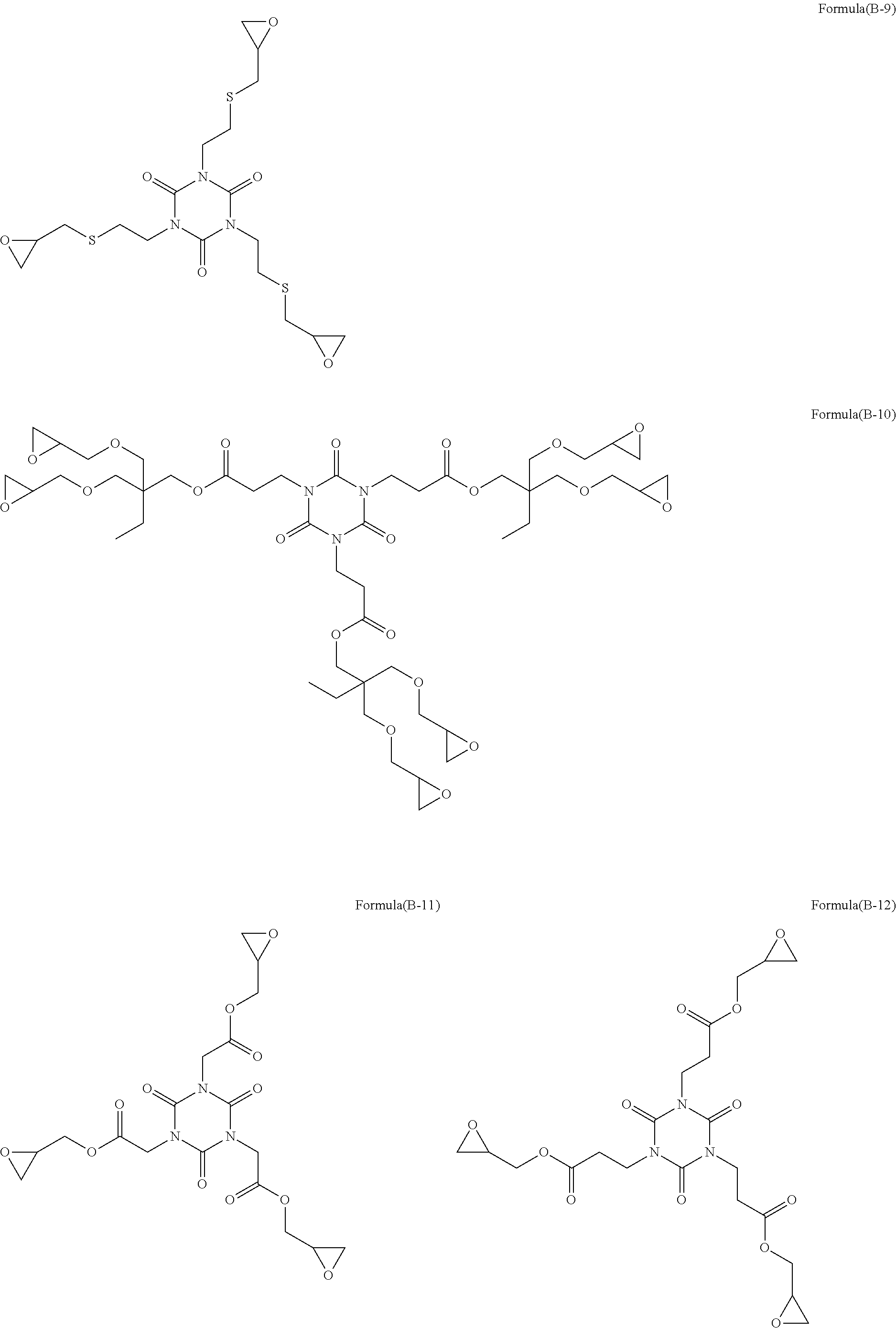 [SMILES: O=c1n(CCSCC2CO2)c(=O)n(CCSCC2CO2)c(=O)n1CCSCC1CO1 ; CCC(COCC1CO1)(COCC1CO1)COC(=O)CCn1c(=O)n(CCC(=O)OCC(CC)(COCC2CO2)COCC2CO2)c(=O)n(CCC(=O)OCC(CC)(COCC2CO2)COCC2CO2)c1=O ; O=C(Cn1c(=O)n(CC(=O)OCC2CO2)c(=O)n(CC(=O)OCC2CO2)c1=O)OCC1CO1 ; O=C(CCn1c(=O)n(CCC(=O)OCC2CO2)c(=O)n(CCC(=O)OCC2CO2)c1=O)OCC1CO1]

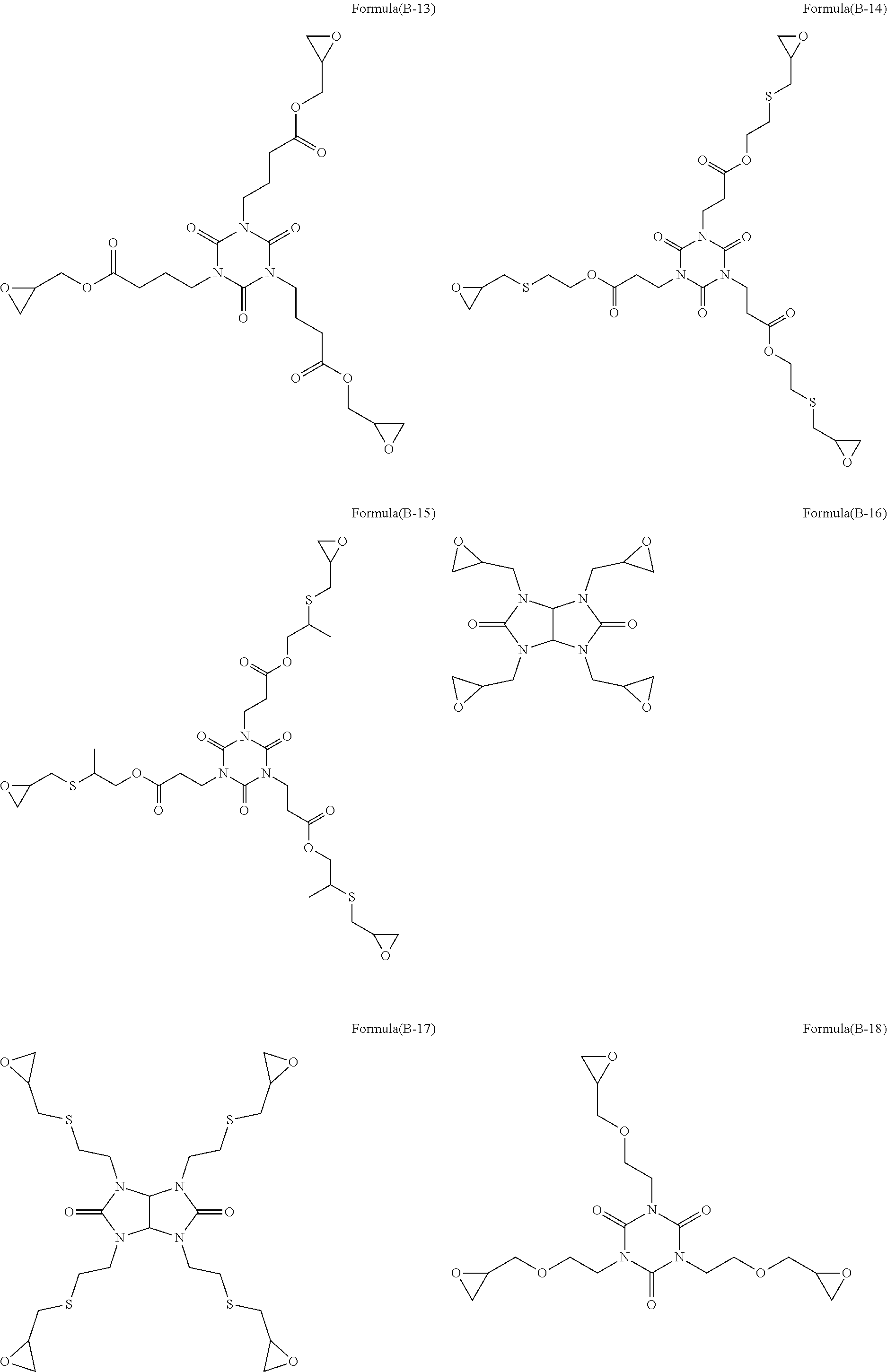 [SMILES: O=C(CCCn1c(=O)n(CCCC(=O)OCC2CO2)c(=O)n(CCCC(=O)OCC2CO2)c1=O)OCC1CO1 ; O=C(CCn1c(=O)n(CCC(=O)OCCSCC2CO2)c(=O)n(CCC(=O)OCCSCC2CO2)c1=O)OCCSCC1CO1 ; CC(COC(=O)CCn1c(=O)n(CCC(=O)OCC(C)SCC2CO2)c(=O)n(CCC(=O)OCC(C)SCC2CO2)c1=O)SCC1CO1 ; O=C1N(CC2CO2)C2C(N1CC1CO1)N(CC1CO1)C(=O)N2CC1CO1 ; O=C1N(CCSCC2CO2)C2C(N1CCSCC1CO1)N(CCSCC1CO1)C(=O)N2CCSCC1CO1 ; O=c1n(CCOCC2CO2)c(=O)n(CCOCC2CO2)c(=O)n1CCOCC1CO1]

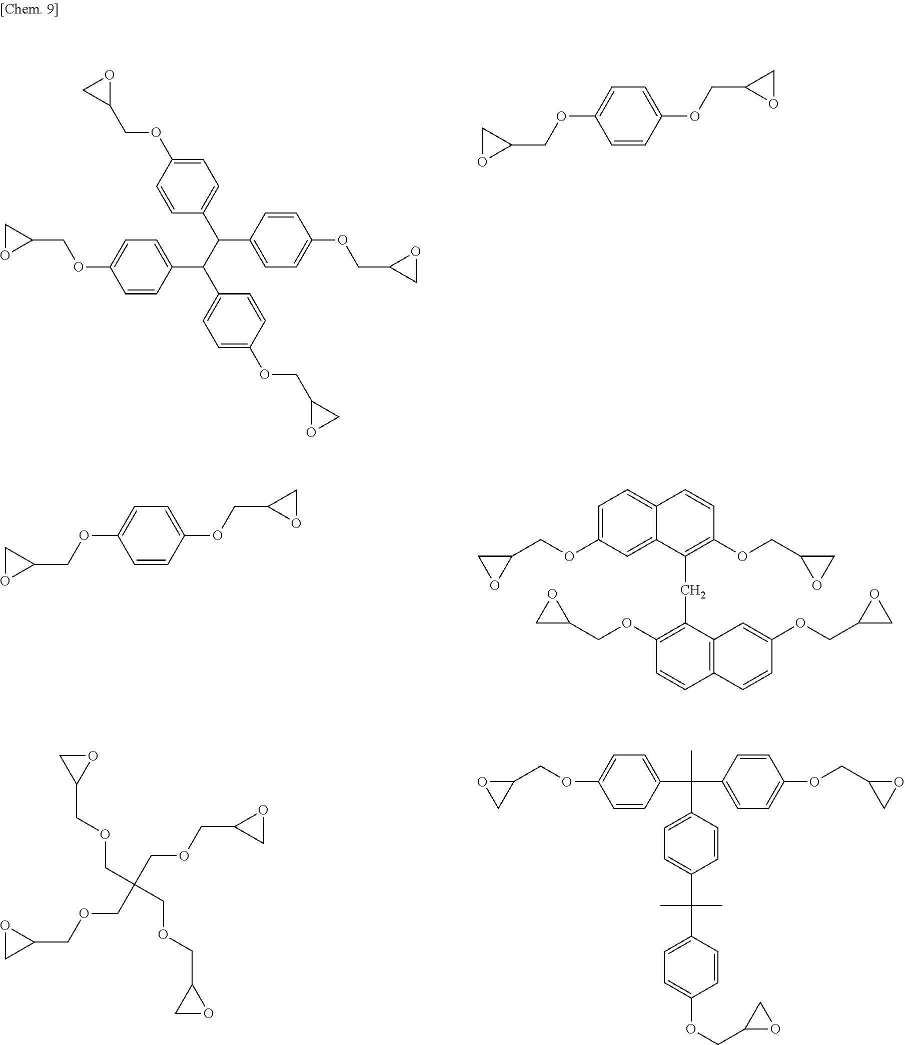 [SMILES: c1cc(C(c2ccc(OCC3CO3)cc2)C(c2ccc(OCC3CO3)cc2)c2ccc(OCC3CO3)cc2)ccc1OCC1CO1 ; c1cc(OCC2CO2)ccc1OCC1CO1 ; c1cc(OCC2CO2)ccc1OCC1CO1 ; c1cc2ccc(OCC3CO3)c(Cc3c(OCC4CO4)ccc4ccc(OCC5CO5)cc34)c2cc1OCC1CO1 ; C(OCC(COCC1CO1)(COCC1CO1)COCC1CO1)C1CO1 ; CC(C)(c1ccc(OCC2CO2)cc1)c1ccc(C(C)(c2ccc(OCC3CO3)cc2)c2ccc(OCC3CO3)cc2)cc1]

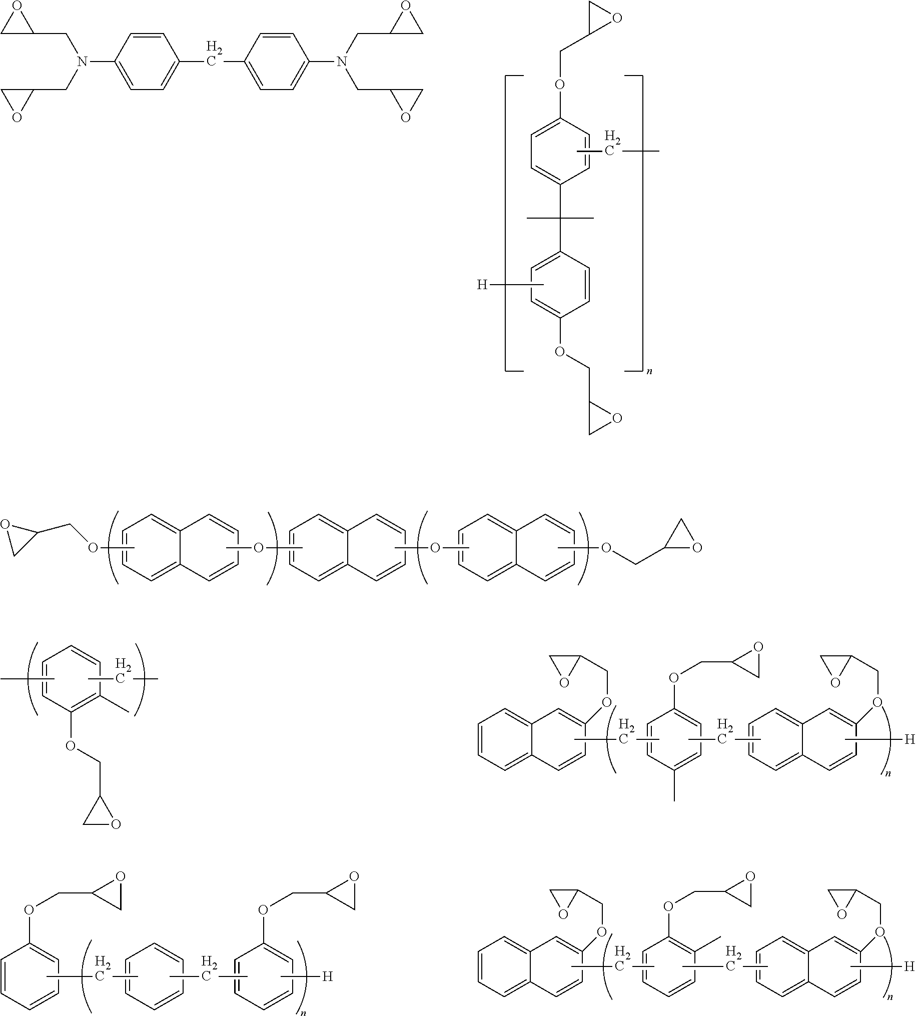

The epoxy group-containing compounds may include a C6-C40 aromatic ring structure. Specific examples thereof include aromatic ring structures derived from, for example, benzene, naphthalene, anthracene, acenaphthene, fluorene, triphenylene, phenalene, phenanthrene, indene, indane, indacene, pyrene, chrysene, perylene, naphthacene, pentacene, coronene, heptacene, benzo[a]anthracene, dibenzophenanthrene, and dibenzo[a,j]anthracene.

The compound (A) may be a reaction product of:
a compound (a) having m quantity of epoxy group(s);
a compound (b) represented by Formula (b) below:

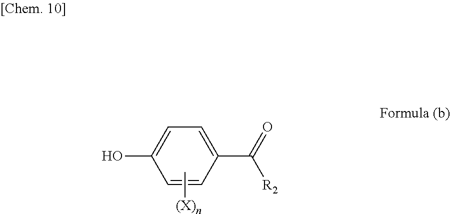

wherein R₂ denotes a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; and n denotes an integer of 0 to 4; and
a compound (c) represented by Formula (c) below:

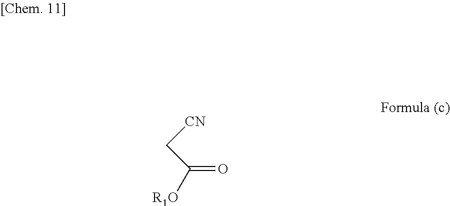

wherein R₁ denotes a hydrogen atom or an optionally substituted C1-C10 alkyl group.

Specific examples of the compounds (a) having m quantity of epoxy group(s) include the compounds illustrated above that include the compounds represented by Formulas (B-1) to (B-18).

Specific examples of the compounds represented by Formula (b) include compounds represented by the following formulas:

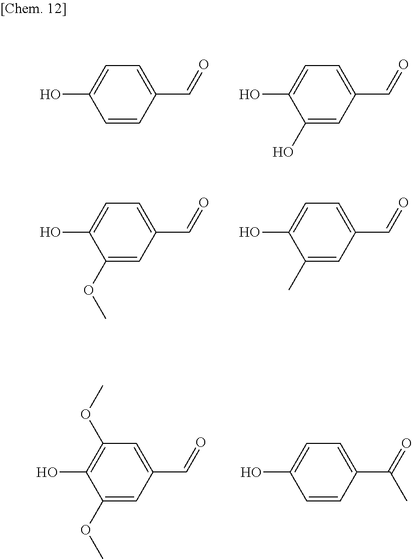

Specific examples of the compounds represented by Formula (c) include compounds represented by the following formulas:

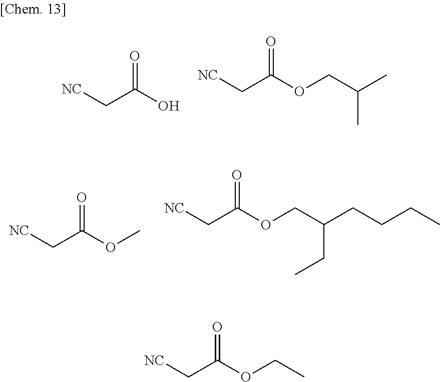

The reaction product may be produced by a known method, for example, by the method described in Examples.

For example, the weight average molecular weight of the compound (A) is in the range of 300 to 3,000.

Moreover, the resist underlayer film-forming composition of the present invention may be a bisphenol-type novolac resin that has a partial structure represented by Formula (1) below:

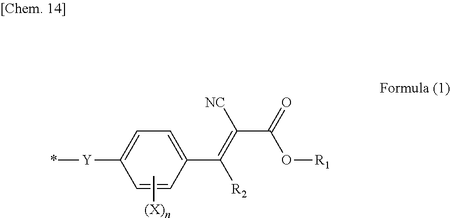

wherein R₁ and R₂ each denote a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; Y denotes a direct bond, an ether bond, a thioether bond, or an ester bond; n denotes an integer of 0 to 4; and * denotes a bond to the bisphenol-type novolac resin.

In the above case, the resist underlayer film-forming composition of the present invention is a composition for forming a resist underlayer film that can be removed with a chemical used for the wet etching of a substrate, such as a copper substrate described later. The resist underlayer film-forming composition of the present invention may be a composition for that purpose, namely, a composition for application to a substrate having copper on a surface.

<Solvent>

The solvent used in the resist underlayer film-forming composition according to the present invention may be any solvent without limitation that can dissolve the compound described above and other components. In particular, it is recommendable to use a combination of solvents commonly utilized in the lithographic process, in consideration of the applicability of the resist underlayer film-forming composition of the present invention used as a uniform solution.

Examples of the solvent include methyl cellosolve acetate, ethyl cellosolve acetate, propylene glycol, propylene glycol monomethyl ether, propylene glycol monoethyl ether, methyl isobutyl carbinol, propylene glycol monobutyl ether, propylene glycol monomethyl ether acetate, propylene glycol monoethyl ether acetate, propylene glycol monopropyl ether acetate, propylene glycol monobutyl ether acetate, toluene, xylene, methyl ethyl ketone, cyclopentanone, cyclohexanone, ethyl 2-hydroxypropionate, ethyl 2-hydroxy-2-methylpropionate, ethyl ethoxyacetate, ethyl hydroxyacetate, methyl 2-hydroxy-3-methylbutanoate, methyl 3-methoxypropionate, ethyl 3-methoxypropionate, ethyl 3-ethoxypropionate, methyl 3-ethoxypropionate, methyl pyruvate, ethyl pyruvate, ethylene glycol monomethyl ether, ethylene glycol monoethyl ether, ethylene glycol monopropyl ether, ethylene glycol monobutyl ether, ethylene glycol monomethyl ether acetate, ethylene glycol monoethyl ether acetate, ethylene glycol monopropyl ether acetate, ethylene glycol monobutyl ether acetate, diethylene glycol dimethyl ether, diethylene glycol diethyl ether, diethylene glycol dipropyl ether, diethylene glycol dibutyl ether propylene glycol monomethyl ether, propylene glycol dimethyl ether, propylene glycol diethyl ether, propylene glycol dipropyl ether, propylene glycol dibutyl ether, ethyl lactate, propyl lactate, isopropyl lactate, butyl lactate, isobutyl lactate, methyl formate, ethyl formate, propyl formate, isopropyl formate, butyl formate, isobutyl formate, amyl formate, isoamyl formate, methyl acetate, ethyl acetate, amyl acetate, isoamyl acetate, hexyl acetate, methyl propionate, ethyl propionate, propyl propionate, isopropyl propionate, butyl propionate, isobutyl propionate, methyl butyrate, ethyl butyrate, propyl butyrate, isopropyl butyrate, butyl butyrate, isobutyl butyrate, ethyl hydroxyacetate, ethyl 2-hydroxy-2-methylpropionate, methyl 3-methoxy-2-methylpropionate, methyl 2-hydroxy-3-methylbutyrate, ethyl methoxyacetate, ethyl ethoxyacetate, methyl 3-methoxypropionate, ethyl 3-ethoxypropionate, ethyl 3-methoxypropionate, 3-methoxybutyl acetate, 3-methoxypropyl acetate, 3-methyl-3-methoxybutyl acetate, 3-methyl-3-methoxybutyl propionate, 3-methyl-3-methoxybutyl butyrate, methyl acetoacetate, toluene, xylene, methyl ethyl ketone, methyl propyl ketone, methyl butyl ketone, 2-heptanone, 3-heptanone, 4-heptanone, cyclohexanone, N,N-dimethylformamide, N-methylacetamide, N,N-dimethylacetamide, N-methylpyrrolidone, 4-methyl-2-pentanol, and γ-butyrolactone. The solvents may be used each alone or in combination of two or more thereof.

Some preferred solvents are propylene glycol monomethyl ether, propylene glycol monomethyl ether acetate, ethyl lactate, butyl lactate, and cyclohexanone. In particular, propylene glycol monomethyl ether and propylene glycol monomethyl ether acetate are preferable.

[Crosslinking Agent]

The resist underlayer film-forming composition of the present invention may include a crosslinking agent component. Examples of the crosslinking agent include melamine-based agents, substituted urea-based agents, and polymers thereof. Crosslinking agents having at least two crosslinking substituents are preferable, with examples including methoxymethylated glycoluril (for example, tetramethoxymethylglycoluril), butoxymethylated glycoluril, methoxymethylated melamine, butoxymethylated melamine, methoxymethylated benzoguanamine, butoxymethylated benzoguanamine, methoxymethylated urea, butoxymethylated urea, and methoxymethylated thiourea. Moreover, condensates of these compounds may also be used.

The crosslinking agent that is used may be a compound that contains in the molecule a crosslinking substituent having an aromatic ring (for example, a benzene ring or a naphthalene ring).

Examples of such a compound include compounds having a partial structure of the following Formula (6), and polymers and oligomers having a repeating unit of the following Formula (7):

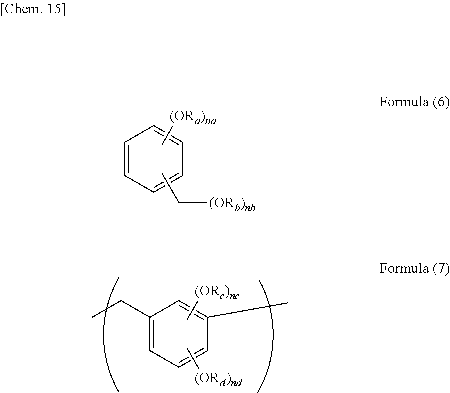

Rₐ, R_{b}, R_{c}, and R_{d} are each a hydrogen atom or a C1-C10 alkyl group; and na, nb, nc, and nd each denote an integer of 0 to 3. Examples of the alkyl groups are the same as enumerated hereinabove.

Examples of the compounds, the polymers, and the oligomers having the Formula (6) or the Formula (7) are illustrated below.

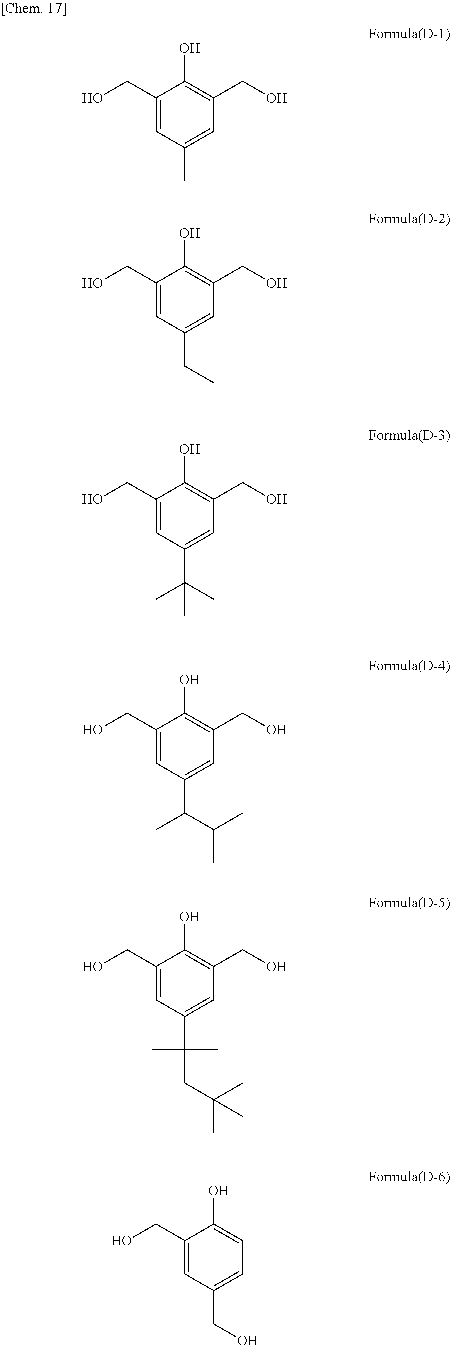 [SMILES: Cc1cc(CO)c(O)c(CO)c1 ; CCc1cc(CO)c(O)c(CO)c1 ; CC(C)(C)c1cc(CO)c(O)c(CO)c1 ; CC(C)C(C)c1cc(CO)c(O)c(CO)c1 ; CC(C)(C)CC(C)(C)c1cc(CO)c(O)c(CO)c1 ; OCc1ccc(O)c(CO)c1]

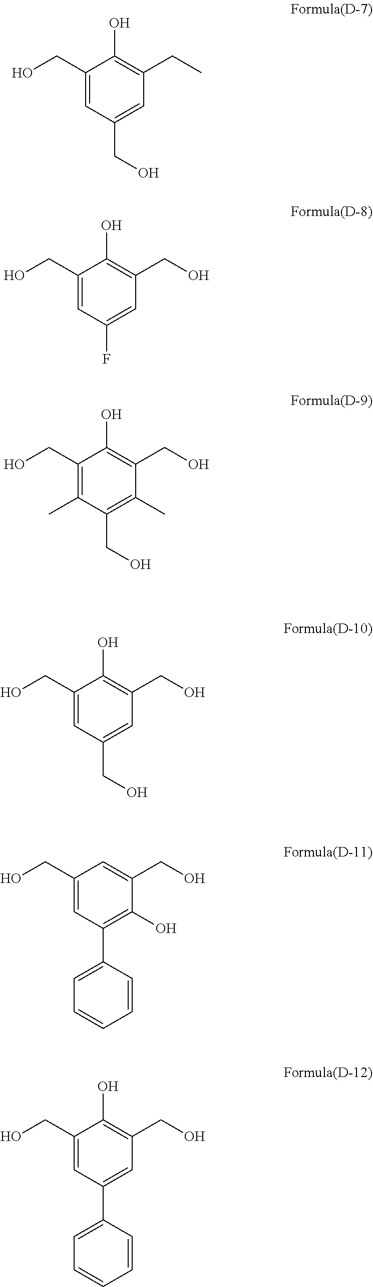 [SMILES: CCc1cc(CO)cc(CO)c1O ; OCc1cc(F)cc(CO)c1O ; Cc1c(CO)c(C)c(CO)c(O)c1CO ; OCc1cc(CO)c(O)c(CO)c1 ; OCc1cc(CO)c(O)c(-c2ccccc2)c1 ; OCc1cc(-c2ccccc2)cc(CO)c1O]

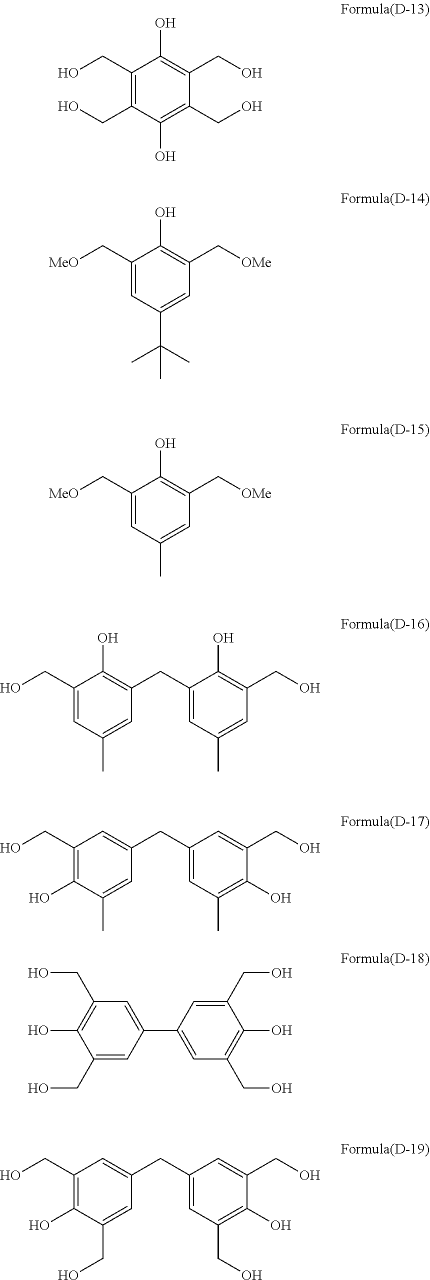

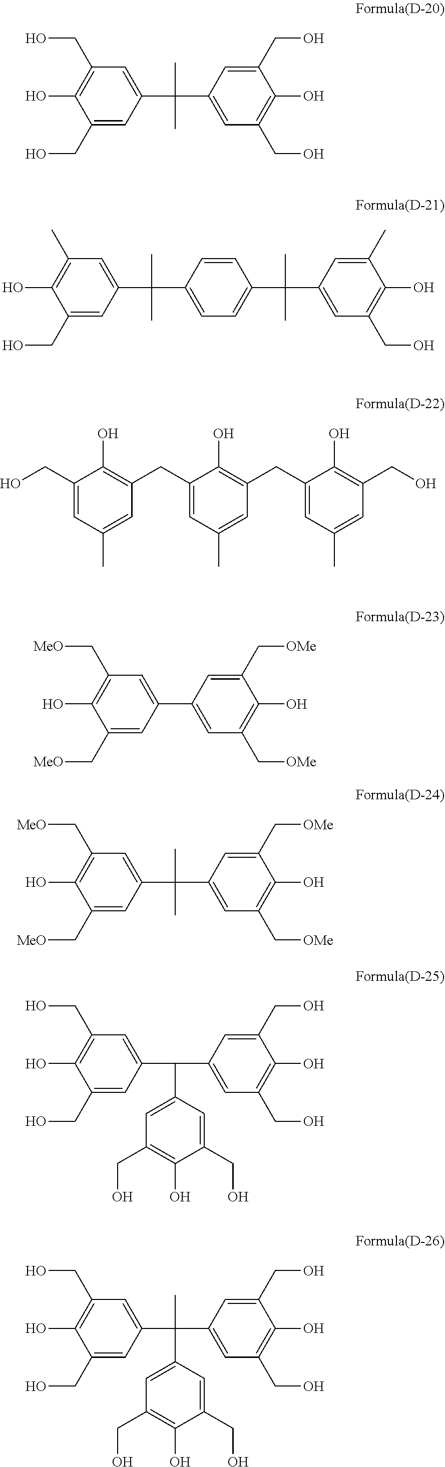

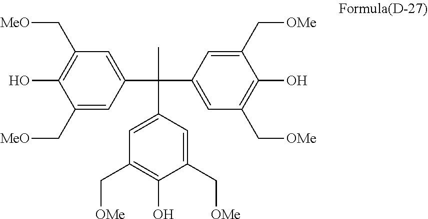 [SMILES: COCc1cc(C(C)(c2cc(COC)c(O)c(COC)c2)c2cc(COC)c(O)c(COC)c2)cc(COC)c1O]

The above compounds are commercially available from ASAHI YUKIZAI CORPORATION or Honshu Chemical Industry Co., Ltd. Of the above crosslinking agents, for example, the compound of Formula (D-24) is available under the product name TM-BIP-A from ASAHI YUKIZAI CORPORATION.

The amount of the crosslinking agent to be added varies depending on such factors as the type of the coating solvent used, the type of the underlying substrate used, the solution viscosity that is required, and the film shape that is required; however, it may be within the rage of 0.001 to 80% by mass, preferably 0.01 to 50% by mass, and more preferably 0.05 to 40% by mass, relative to the total solid content. The above crosslinking agent may cause crosslinking reaction via self-condensation; however, when the reaction product of the present invention has a crosslinking substituent, the crosslinking agent may undergo a crosslinking reaction with the crosslinking substituent.

[Acid and/or Acid Generator]

The resist underlayer film-forming composition of the present invention may contain an acid and/or an acid generator.

Examples of the acid include p-toluenesulfonic acid, trifluoromethanesulfonic acid, pyridinium trifluoromethanesulfonate, pyridinium p-toluenesulfonate, pyridinium phenolsulfonate, salicylic acid, 5-sulfosalicylic acid, 4-phenolsulfonic acid, camphorsulfonic acid, 4-chlorobenzenesulfonic acid, benzenedisulfonic acid, 1-naphthalenesulfonic acid, citric acid, benzoic acid, hydroxybenzoic acid, and naphthalenecarboxylic acid.

The acids may be used each alone or in combination of two or more thereof. The amount thereof is usually within the range of 0.0001 to 20% by mass, preferably 0.0005 to 10% by mass, and more preferably 0.01 to 3% by mass, relative to the total solid content.

Examples of the acid generator include thermal acid generators and photo acid generators.

Examples of the thermal acid generator include pyridinium trifluoromethanesulfonate, pyridinium p-toluenesulfonate, pyridinium phenolsulfonate, 2,4,4,6-tetrabromocyclohexadienone, benzoin tosylate, 2-nitrobenzyl tosylate, and other organic sulfonic acid alkyl esters.

The photo acid generator generates an acid, when a resist is exposed to light, thereby allowing the acidity of the underlayer film to be adjusted. The use of the photo acid generator is an approach to adjusting the acidity of the underlayer film to the acidity of a resist layer that is formed thereon. Moreover, the shape of a pattern formed in the upper resist layer may be controlled by the adjustment of the acidity of the underlayer film.

Examples of the photo acid generator used in the resist underlayer film-forming composition of the present invention include onium salt compounds, sulfonimide compounds, and disulfonyldiazomethane compounds.

Examples of the onium salt compound include iodonium salt compounds, such as diphenyliodonium hexafluorophosphate, diphenyliodonium trifluoromethanesulfonate, diphenyliodonium nonafluoro-n-butanesulfonate, diphenyliodonium perfluoro-n-octanesulfonate, diphenyliodonium camphorsulfonate, bis(4-tert-butylphenyl)iodonium camphorsulfonate, and bis(4-tert-butylphenyl)iodonium trifluoromethanesulfonate; and sulfonium salt compounds, such as triphenylsulfonium hexafluoroantimonate, triphenylsulfonium nonafluoro-n-butanesulfonate, triphenylsulfonium camphorsulfonate, and triphenylsulfonium trifluoromethanesulfonate.

Examples of the sulfonimide compound include N-(trifluoromethanesulfonyloxy)succinimide, N-(nonafluoro-n-butanesulfonyloxy)succinimide, N-(camphorsulfonyloxy)succinimide, and N-(trifluoromethanesulfonyloxy)naphthalimide.

Examples of the disulfonyldiazomethane compound include bis(trifluoromethylsulfonyl)diazomethane, bis(cyclohexylsulfonyl)diazomethane, bis(phenylsulfonyl)diazomethane, bis(p-toluenesulfonyl)diazomethane, bis(2,4-dimethylbenzenesulfonyl)diazomethane, and methylsulfonyl-p-toluenesulfonyldiazomethane.

The acid generators may be used each alone or in combination of two or more thereof.

When an acid generator is used, the proportion thereof is usually within the range of 0.0001 to 20% by mass, preferably 0.0005 to 10% by mass, and more preferably 0.01 to 3% by mass, relative to 100 parts by mass of the solid content in the resist underlayer film-forming composition.

[Additional Components]

A surfactant may be added to the resist underlayer film-forming composition of the present invention in order to prevent the occurrence of defects, such as pinholes and striation, and to further enhance the applicability with respect to uneven surfaces. Examples of the surfactant include nonionic surfactants, for example, polyoxyethylene alkyl ethers, such as polyoxyethylene lauryl ether, polyoxyethylene stearyl ether, polyoxyethylene cetyl ether, and polyoxyethylene oleyl ether, polyoxyethylene alkyl allyl ethers, such as polyoxyethylene octyl phenol ether and polyoxyethylene nonyl phenol ether, polyoxyethylene/polyoxypropylene block copolymers, sorbitan fatty acid esters, such as sorbitan monolaurate, sorbitan monopalmitate, sorbitan monostearate, sorbitan monooleate, sorbitan trioleate, and sorbitan tristearate, and polyoxyethylene sorbitan fatty acid esters, such as polyoxyethylene sorbitan monolaurate, polyoxyethylene sorbitan monopalmitate, polyoxyethylene sorbitan monostearate, polyoxyethylene sorbitan trioleate, and polyoxyethylene sorbitan tristearate; fluorine surfactants, such as EFTOP series EF301, EF303, and EF352 (product names, manufactured by Tochem Products Co., Ltd.), MEGAFACE series F171, F173, R-40, R-40N, and R-40LM (product names, manufactured by DIC CORPORATION), FLUORAD series FC430 and FC431 (product names, manufactured by Sumitomo 3M Ltd.), and ASAHI GUARD AG710 and SURFLON series S-382, SC101, SC102, SC103, SC104, SC105, and SC106 (product names, manufactured by AGC Inc.); and organosiloxane polymer KP341 (manufactured by Shin-Etsu Chemical Co., Ltd.). The amount of the surfactant is usually within the range of 2.0% by mass or less, and preferably 1.0% by mass or less, relative to the total solid content in the resist underlayer film material. The surfactants may be used each alone or in combination of two or more thereof. When a surfactant is used, the proportion thereof is within the range of 0.0001 to 5 parts by mass, or 0.001 to 1 part by mass, or 0.01 to 0.5 parts by mass, with respect to 100 parts by mass of the solid content in the resist underlayer film-forming composition.

Additives, such as light absorbers, rheology modifiers, and adhesion aids, may be added to the resist underlayer film-forming composition of the present invention. Rheology modifiers are effective for enhancing the fluidity of the underlayer film-forming composition. Adhesion aids are effective for enhancing the adhesion between the underlayer film and either a semiconductor substrate or a resist.

Some exemplary light absorbers that may be suitably used are commercially available light absorbers described in “Kougyouyou Shikiso no Gijutsu to Shijou (Technology and Market of Industrial Dyes)” (CMC Publishing Co., Ltd.) and “Senryou Binran (Dye Handbook)” (edited by The Society of Synthetic Organic Chemistry, Japan), such as, for example, C. I. Disperse Yellow 1, 3, 4, 5, 7, 8, 13, 23, 31, 49, 50, 51, 54, 60, 64, 66, 68, 79, 82, 88, 90, 93, 102, 114, and 124; C. I. Disperse Orange 1, 5, 13, 25, 29, 30, 31, 44, 57, 72, and 73; C. I. Disperse Red 1, 5, 7, 13, 17, 19, 43, 50, 54, 58, 65, 72, 73, 88, 117, 137, 143, 199, and 210; C. I. Disperse Violet 43; C. I. Disperse Blue 96; C. I. Fluorescent Brightening Agent 112, 135, and 163; C. I. Solvent Orange 2 and 45; C. I. Solvent Red 1, 3, 8, 23, 24, 25, 27, and 49; C. I. Pigment Green 10; and C. I. Pigment Brown 2. The light absorber is usually added in a proportion of 10% by mass or less, preferably 5% by mass or less, relative to the total solid content in the resist underlayer film-forming composition.

The rheology modifier may be added mainly to enhance the fluidity of the resist underlayer film-forming composition and thereby, particularly in the baking step, to enhance the uniformity in thickness of the resist underlayer film and to increase the filling performance of the resist underlayer film-forming composition with respect to the inside of holes. Specific examples thereof include phthalic acid derivatives, such as dimethyl phthalate, diethyl phthalate, diisobutyl phthalate, dihexyl phthalate, and butyl isodecyl phthalate; adipic acid derivatives, such as di-n-butyl adipate, diisobutyl adipate, diisooctyl adipate, and octyl decyl adipate; maleic acid derivatives, such as di-n-butyl maleate, diethyl maleate, and dinonyl maleate; oleic acid derivatives, such as methyl oleate, butyl oleate, and tetrahydrofurfuryl oleate; and stearic acid derivatives, such as n-butyl stearate and glyceryl stearate. The rheology modifier is usually added in a proportion of less than 30% by mass, relative to the total solid content in the resist underlayer film-forming composition.

The adhesion aid may be added mainly to enhance the adhesion between the resist underlayer film-forming composition and either a substrate or a resist and thereby to prevent the detachment of the resist particularly during development. Specific examples thereof include chlorosilanes, such as trimethylchlorosilane, dimethylmethylolchlorosilane, methyldiphenylchlorosilane, and chloromethyldimethylchlorosilane; alkoxysilanes, such as trimethylmethoxysilane, dimethyldiethoxysilane, methyldimethoxysilane, dimethylmethylolethoxysilane, diphenyldimethoxysilane, and phenyltriethoxysilane; silazanes, such as hexamethyldisilazane, N,N′-bis(trimethylsilyl)urea, dimethyltrimethylsilylamine, and trimethylsilylimidazole; silanes, such as methyloltrichlorosilane, γ-chloropropyltrimethoxysilane, γ-aminopropyltriethoxysilane, and γ-glycidoxypropyltrimethoxysilane; heterocyclic compounds, such as benzotriazole, benzimidazole, indazole, imidazole, 2-mercaptobenzimidazole, 2-mercaptobenzothiazole, 2-mercaptobenzoxazole, urazole, thiouracil, mercaptoimidazole, and mercaptopyrimidine; and urea or thiourea compounds, such as 1,1-dimethylurea and 1,3-dimethylurea. The adhesion aid is usually added in a proportion of less than 5% by mass, preferably less than 2% by mass, relative to the total solid content in the resist underlayer film-forming composition.

The solid content in the resist underlayer film-forming composition according to the present invention is usually within the range of 0.1 to 70% by mass, 0.1 to 60% by mass, 0.1 to 50% by mass, 0.1 to 40% by mass, 0.1 to 30% by mass, 0.1 to 20% by mass, 0.1 to 10% by mass, 0.1 to 5% by mass, 0.1 to 3% by mass, or 0.1 to 2% by mass. The solid content is the proportion of all the components constituting the resist underlayer film-forming composition except the solvent. The proportion of the reaction product in the solid content is, with increasing order of preference, within the range of 1 to 100% by mass, 1 to 99.9% by mass, 50 to 99.9% by mass, 50 to 95% by mass, or 50 to 90% by mass.

One of the measures for evaluating whether a resist underlayer film-forming composition is in a state of uniform solution is to observe the passing property of the composition through a predetermined microfilter. The resist underlayer film-forming composition according to the present invention can be passed through a microfilter having a pore size of 0.1 μm and exhibits a uniform solution state.

Examples of the microfilter material include fluororesins, such as PTFE (polytetrafluoroethylene) and PFA (tetrafluoroethylene/perfluoroalkyl vinyl ether copolymer), PE (polyethylene), UPE (ultrahigh molecular weight polyethylene), PP (polypropylene), PSF (polysulfone), PES (polyethersulfone), and nylon, with PTFE (polytetrafluoroethylene) being preferable.

[Substrate]

Examples of the substrate used in the manufacturing of semiconductor devices in the present invention include silicon wafer substrates, silicon/silicon dioxide coated substrates, silicon nitride substrates, glass substrates, ITO substrates, polyimide substrates, and low-dielectric constant material (low-k material)-coated substrates.

The field of three-dimensional packaging in the semiconductor manufacturing has recently started to adopt the FOWLP process for the purposes of high-speed response and power saving by shortening of the wiring length between semiconductor chips. In the RDL (redistribution) process that fabricates wires between semiconductor chips, copper (Cu) is used as a wiring member. As the copper wiring design becomes finer, the application of an anti-reflective film (a resist underlayer film-forming composition) becomes necessary. The resist underlayer film-forming composition according to the present invention may be suitably applied even to a substrate having copper on a surface.

[Resist Underlayer Film and Method for Manufacturing Semiconductor Devices]

Hereinbelow, the resist underlayer film from the resist underlayer film-forming compositions according to the present invention, and the method for manufacturing semiconductor devices will be described.

The resist underlayer film-forming composition of the present invention is applied onto the substrate described above used for manufacturing a semiconductor device (for example, a substrate having copper on a surface) with an appropriate application technique, such as a spinner or a coater, and the coating is baked to form a resist underlayer film.

The baking conditions are appropriately selected from baking temperatures of 80° C. to 400° C., and amounts of baking time of 0.3 to 60 minutes. The baking temperature is preferably 150° C. to 350° C., and the baking time is preferably 0.5 to 2 minutes. Here, the film thickness of the underlayer film that is formed is, for example, 10 to 1000 nm, or 20 to 500 nm, or 30 to 400 nm, or 50 to 300 nm.

Moreover, an inorganic resist underlayer film (a hard mask) may be formed on the organic resist underlayer film according to the present invention. For example, such a hard mask may be formed by spin coating a silicon-containing resist underlayer film (inorganic resist underlayer film)-forming composition described in WO 2009/104552 A1, or by CVD of a Si-based inorganic material film.

Next, a resist film, for example, a photoresist layer is formed on the resist underlayer film. The photoresist layer may be formed by a well-known method similar to removing the solvent from a coating film of the resist underlayer film-forming composition, specifically, by applying a photoresist composition solution onto the underlayer film followed by baking. For example, the film thickness of the photoresist is within the range of 50 to 10.000 nm or 100 to 2,000 nm.

The photoresist applied onto the resist underlayer film is not particularly limited as long as it is sensitive to light used in the exposure. Negative photoresists and positive photoresists may be used. Examples include positive photoresists composed of a novolac resin and a 1,2-naphthoquinonediazide sulfonic acid ester, chemically amplified photoresists composed of a photo acid generator and a binder having a group that is decomposed by an acid to increase the alkali dissolution rate, chemically amplified photoresists composed of an alkali-soluble binder, a photo acid generator, and a low-molecular compound that is decomposed by an acid to increase the alkali dissolution rate of the photoresist, and chemically amplified photoresists composed of a photo acid generator, a binder having a group that is decomposed by an acid to increase the alkali dissolution rate, and a low-molecular compound that is decomposed by an acid to increase the alkali dissolution rate of the photoresist. Specific examples include those available under the product names of APEX-E from Shipley, PAR 710 from Sumitomo Chemical Co., Ltd., and SEPR 430 from Shin-Etsu Chemical Co., Ltd. Examples further include fluorine-containing polymer-based photoresists described in Proc. SPIE, Vol. 3999, 330-334 (2000), Proc. SPIE, Vol. 3999, 357-364 (2000), and Proc. SPIE, Vol. 3999, 365-374 (2000).

Next, a resist pattern is formed by a light or electron beam irradiation and development. First, the resist is exposed through a predetermined mask. For example, a near ultraviolet ray, a far ultraviolet ray, or an extreme ultraviolet ray (e.g., EUV (wavelength: 13.5 nm)) may be used for the exposure. Specifically, for example, use may be made of i-radiation (wavelength: 365 nm), KrF excimer laser beam (wavelength: 248 nm), ArF excimer laser beam (wavelength: 193 nm), or F2 excimer laser beam (wavelength: 157 nm). Of these, i-radiation (wavelength: 365 nm) is preferable. After the exposure, post-exposure baking may be performed as required. The post-exposure baking is performed under conditions appropriately selected from baking temperatures of 70° C. to 150° C., and amounts of baking time of 0.3 to 10 minutes.

In the present invention, the above resist, namely, the photoresist may be replaced by an electron beam lithographic resist. The electron beam resists may be negative or positive. Examples include chemically amplified resists composed of an acid generator and a binder having a group that is decomposed by an acid to give rise to a change in alkali dissolution rate, chemically amplified resists composed of an alkali-soluble binder, an acid generator, and a low-molecular compound that is decomposed by an acid to change the alkali dissolution rate of the resist, chemically amplified resists composed of an acid generator, a binder having a group that is decomposed by an acid to give rise to a change in alkali dissolution rate, and a low-molecular compound that is decomposed by an acid to change the alkali dissolution rate of the resist, non-chemically amplified resists composed of a binder having a group that is decomposed by an electron beam to give rise to a change in alkali dissolution rate, and non-chemically amplified resists composed of a binder having a moiety that is cleaved by an electron beam to give rise to a change in alkali dissolution rate. The electron beam resist may be patterned using an electron beam as the irradiation source in the same manner as when the photoresist is used.

Next, the resist is developed with a developer. When, for example, the resist is a positive photoresist, the portions of the photoresist that have been exposed are removed to leave a photoresist pattern.

Examples of the developers include alkaline aqueous solutions, for example, aqueous solutions of alkali metal hydroxides, such as potassium hydroxide and sodium hydroxide, aqueous solutions of quaternary ammonium hydroxides, such as tetramethylammonium hydroxide, tetraethylammonium hydroxide, and choline, and aqueous solutions of amines, such as ethanolamine, propylamine, and ethylenediamine. Moreover, additives, such as surfactants, may be added to the developers. The development conditions are appropriately selected from temperatures of 5 to 50° C., and amounts of time of 10 to 600 seconds.

In the present invention, an organic underlayer film (a lower layer) may be formed on a substrate, thereafter an inorganic underlayer film (an intermediate layer) may be formed thereon, and further a photoresist (an upper layer) may be formed thereon. Even in the case where the photoresist is designed with a narrow pattern width and is formed with a small thickness to avoid collapsing of the pattern, the configuration described above in combination with selection of appropriate etching gases allows the substrate to be processed as designed. For example, processing may be performed into the resist underlayer films using as the etching gas a fluorine-containing gas capable of etching the photoresist at a sufficiently high rate; the substrate may be processed using as the etching gas a fluorine-containing gas capable of etching the inorganic underlayer film at a sufficiently high rate; and further the substrate may be processed using as the etching gas an oxygen-containing gas capable of etching the organic underlayer film at a sufficiently high rate.

After the photoresist has been patterned as described above, the inorganic underlayer film is removed using the pattern as a protective film, and thereafter, the organic underlayer film is removed using, as a protective film, the film consisting of the patterned photoresist and inorganic underlayer film. Lastly, the semiconductor substrate is processed using, as a protective film, the patterned inorganic underlayer film and organic underlayer film.

First, the portions of the inorganic underlayer film exposed between the photoresist are removed by dry etching to expose the semiconductor substrate. The dry etching of the inorganic underlayer film may be performed using such a gas as tetrafluoromethane (CF₄), perfluorocyclobutane (C₄F₈), perfluoropropane (C₃F₈), trifluoromethane, carbon monoxide, argon, oxygen, nitrogen, sulfur hexafluoride, difluoromethane, nitrogen trifluoride, chlorine trifluoride, chlorine, trichloroborane, or dichloroborane. A halogen-containing gas is preferably used in the dry etching of the inorganic underlayer film, and a fluorine-containing gas is more preferably used. Examples of the fluorine-containing gases include tetrafluoromethane (CF₄), perfluorocyclobutane (C₄F₈), perfluoropropane (C₃F₈), trifluoromethane, and difluoromethane (CH₂F₂).

Subsequently, the organic underlayer film is removed using, as a protective film, the film consisting of the patterned photoresist and inorganic underlayer film.

The organic underlayer film is often removed by dry etching using an oxygen-containing gas for the reason that the inorganic underlayer film containing a large amount of silicon atoms is hardly removed by dry etching with an oxygen-containing gas.

Lastly, the semiconductor substrate is processed. The semiconductor substrate is preferably processed by dry etching with a fluorine-containing gas.

Examples of the fluorine-containing gas include tetrafluoromethane (CF₄), perfluorocyclobutane (C₄F₈), perfluoropropane (C₃F₈), trifluoromethane, and difluoromethane (CH₂F₂).

Before the formation of the photoresist, an organic anti-reflective film may be formed as an upper layer on the resist underlayer films. The anti-reflective coating composition used herein is not particularly limited and may be appropriately selected from those conventionally used in the lithographic processes. The anti-reflective film may be formed by a conventional method, for example, by application with a spinner or a coater, followed by baking.

The resist underlayer film formed from the resist underlayer film-forming composition sometimes shows absorption with respect to the light used in the lithographic process, depending on the wavelength of the light. In such cases, the film can function as an anti-reflective film to effectively prevent the reflection of light from the substrate. Moreover, the underlayer film formed from the resist underlayer film-forming composition of the present invention can also function as a hard mask. The underlayer film of the present invention may be used, for example, as a layer for preventing the interaction between a substrate and a photoresist, as a layer having a function to prevent adverse effects on a substrate by a material used in a photoresist or by a substance generated during the exposure of a photoresist, as a layer having a function to prevent the diffusion of substances generated from a substrate during heating and baking into an upper photoresist layer, and as a barrier layer for reducing the poisoning effects on a photoresist layer by a semiconductor substrate dielectric layer.

Moreover, the underlayer film formed from the resist underlayer film-forming composition may be used as a filling material that is applied to a via-hole substrate used in the dual damascene process and can fill the holes without gap. Furthermore, the underlayer film may also be used as a flattening material for flattening the surface of an irregular semiconductor substrate.

Meanwhile, wet etching with chemicals is a removal technique alternative to dry etching removal and is studied for the purposes of simplifying the process steps, reducing the substrate damage, and saving costs. However, resist underlayer films formed from the conventional resist underlayer film-forming compositions basically need to be solvent-resistant cured films in order to avoid mixing with a resist that is applied thereto. Moreover, the cured films essentially need to be resistant to developers that are used to develop resists into resist patterns. The conventional techniques have difficulties in providing cured films that are insoluble in resist solvents and developers and are soluble exclusively in wet etching solutions. In contrast, the resist underlayer film-forming composition according to the present invention makes it possible to form a resist underlayer film that can be etched (removed) with a wet etching solution.

For example, the wet etching solution preferably includes an organic solvent and may contain an acidic compound or a basic compound. Examples of the organic solvent include dimethylsulfoxide, dimethylformamide, dimethylacetamide, N-methylpyrrolidone, N-ethylpyrrolidone, ethylene glycol, propylene glycol, and diethylene glycol dimethyl ether. Examples of the acidic compound include inorganic acids and organic acids. Examples of the inorganic acids include hydrochloric acid, sulfuric acid, nitric acid, and phosphoric acid. Examples of the organic acids include p-toluenesulfonic acid, trifluoromethanesulfonic acid, salicylic acid, 5-sulfosalicylic acid, 4-phenolsulfonic acid, camphorsulfonic acid, 4-chlorobenzenesulfonic acid, benzenedisulfonic acid, 1-naphthalenesulfonic acid, acetic acid, propionic acid, trifluoroacetic acid, citric acid, benzoic acid, hydroxybenzoic acid, and naphthalenecarboxylic acid. Examples of the basic compound include inorganic bases and organic bases. Examples of the inorganic bases include alkali metal hydroxides, such as sodium hydroxide and potassium hydroxide; quaternary ammonium hydroxides, such as tetramethylammonium hydroxide, tetraethylammonium hydroxide, and choline; and amines, such as ethanolamine, propylamine, diethylaminoethanol, and ethylenediamine. Moreover, the wet etching solution may contain a single organic solvent or may include two or more organic solvents in combination. Furthermore, the acidic compounds or the basic compounds may be used each alone or in combination of two or more thereof. The amount of the acidic compound or basic compound to be added is within the range of 0.01 to 20% by weight, preferably 0.1 to 5% by weight, and particularly preferably 0.2 to 1% by weight, relative to the wet etching solution. The wet etching solution is preferably a solution of an organic solvent containing a basic compound and is particularly preferably a mixed solution including dimethylsulfoxide and tetramethylammonium hydroxide.

The field of three-dimensional packaging in the semiconductor manufacturing has recently started to adopt the FOWLP (Fan-Out Wafer Level Package) process. The resist underlayer film may be applied in the RDL (redistribution) process of forming copper wires.

A typical RDL process is described below, but the process is not limited thereto. First, a photosensitive insulating film is formed on a semiconductor chip and is patterned by light irradiation (exposure) and development to form semiconductor chip electrode openings. Subsequently, sputtering is performed to form a copper seed layer on which copper wiring that is a wiring member will be formed in the plating step. Then, a resist underlayer film and a photoresist layer are sequentially formed, and the resist is patterned by light irradiation and development. Unnecessary portions of the resist underlayer film are removed by dry etching, and electrolytical plating with copper is carried out on the copper seed layer exposed between the resist pattern to form copper wiring, which will serve as a first wiring layer. Then, the resist, the resist underlayer film, and the copper seed layer that are no longer necessary are removed by dry etching, wet etching, or both. Then, the copper wiring layer thus formed is covered again with an insulating film. Thereafter, a copper seed layer, a resist underlayer film, and a resist are formed in this order, and resist patterning, resist underlayer film removal, and copper plating are carried out to form a second copper wiring layer. After the desired copper wiring is formed by repeating these steps, electrode leading-out bumps are finished.

The resist underlayer film-forming composition according to the present invention can form a resist underlayer film that is removable by wet etching, and is therefore particularly suited for use as a resist underlayer film in the RDL process to simplify the process steps and reduce damage to the workpiece substrate.

### EXAMPLES

Next, the present invention will be described in more detail with reference to Examples. However, the scope of the present invention is not limited to such Examples.

The following are the apparatus and conditions used in the measurement of the weight average molecular weight of polymers obtained in Synthesis Examples below.
Apparatus: HLC-8320 GPC manufactured by TOSOH CORPORATION
GPC columns: Shodex [registered trademark] Asahipak [registered trademark] (Showa Denko K.K.)
Column temperature: 40° C.
Flow rate: 0.35 mL/min
Eluent: Tetrahydrofuran (THF)
Standard samples: Polystyrenes (TOSOH CORPORATION)

### Synthesis Example 1

A reaction flask was charged with 5.00 g of a triazinetrione-type epoxy compound (product name: TEPIC, manufactured by Nissan Chemical Corporation, epoxy functionality: 10.03 eq./kg), 6.12 g of 4-hydroxybenzaldehyde, 0.43 g of tetrabutylphosphonium bromide, and 46.17 g of propylene glycol monomethyl ether. In a nitrogen atmosphere, the materials were heated at an internal temperature of 105° C. for 24 hours, while stirring. Subsequently, the reaction solution was cooled to room temperature, and a solution of 5.01 g of methyl cyanoacetate in 20.06 g of propylene glycol monomethyl ether was added to the system. The mixture was heated at an internal temperature of 105° C., for 4 hours, while stirring in a nitrogen atmosphere. The reaction product obtained corresponded to Formula (1-1) and had a weight average molecular weight Mw of 970 as measured by GPC relative to polystyrene.

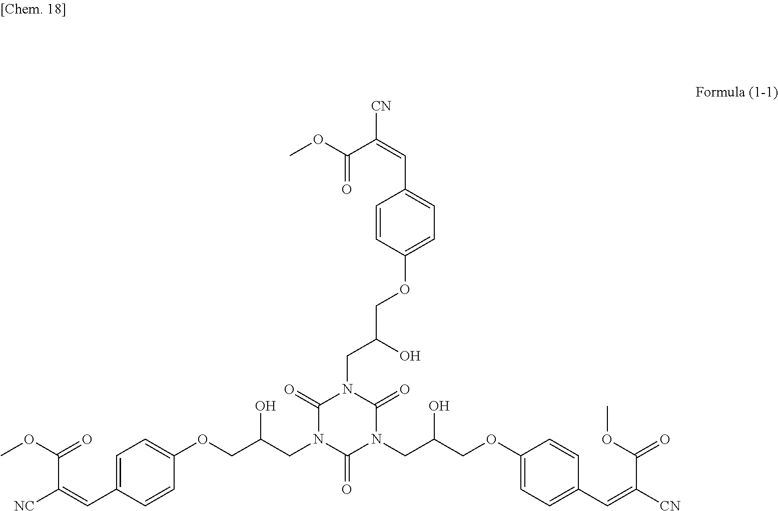 [SMILES: COC(=O)/C(C#N)=C\c1ccc(OCC(O)Cn2c(=O)n(CC(O)COc3ccc(/C=C(/C#N)C(=O)OC)cc3)c(=O)n(CC(O)COc3ccc(/C=C(/C#N)C(=O)OC)cc3)c2=O)cc1]

### Synthesis Example 2

A reaction flask was charged with 5.00 g of a triazinetrione-type epoxy compound (product name: TEPIC, manufactured by Nissan Chemical Corporation, epoxy functionality: 10.03 eq./kg), 6.12 g of 4-hydroxybenzaldehyde, 0.43 g of tetrabutylphosphonium bromide, and 46.17 g of propylene glycol monomethyl ether. In a nitrogen atmosphere, the materials were heated at an internal temperature of 105° C. for 24 hours, while stirring. Subsequently, the reaction solution was cooled to room temperature, and a solution of 4.35 g of cyanoacetic acid in 17.39 g of propylene glycol monomethyl ether was added to the system. The mixture was heated at an internal temperature of 105° C., for 4 hours, while stirring in a nitrogen atmosphere. The reaction product obtained corresponded to Formula (1-2) and had a weight average molecular weight Mw of 800 as measured by GPC relative to polystyrene.

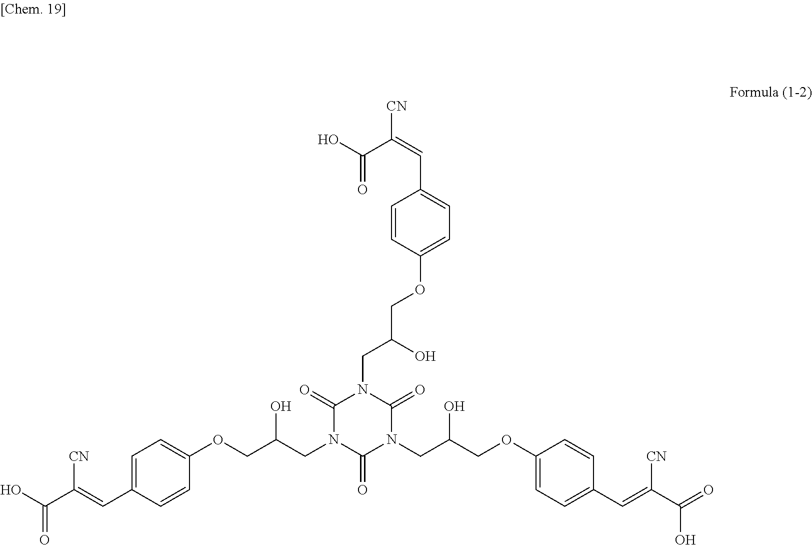 [SMILES: N#C/C(=C/c1ccc(OCC(O)Cn2c(=O)n(CC(O)COc3ccc(/C=C(\C#N)C(=O)O)cc3)c(=O)n(CC(O)COc3ccc(/C=C(\C#N)C(=O)O)cc3)c2=O)cc1)C(=O)O]

### Synthesis Example 3

A reaction flask was charged with 15.00 g of a phenol novolac-type epoxy resin (product name: DEN, manufactured by The Dow Chemical Company, epoxy functionality: 5.55 eq./kg), 10.17 g of 4-hydroxybenzaldehyde, 1.41 g of tetrabutylphosphonium bromide, and 39.87 g of propylene glycol monomethyl ether. In a nitrogen atmosphere, the materials were heated under reflux for 24 hours. Subsequently, a solution of 5.50 g of malononitrile in 34.99 g of propylene glycol monomethyl ether was added to the system. The mixture was heated under reflux for 4 hours. The reaction product obtained corresponded to Formula (1-3) and had a weight average molecular weight Mw of 2,100 as measured by GPC relative to polystyrene.

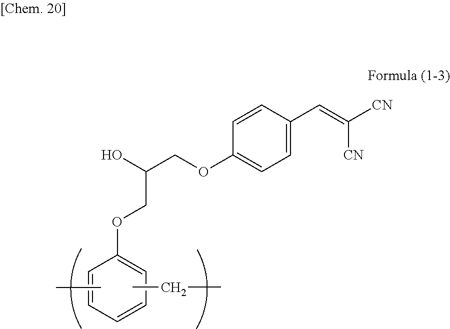

### Synthesis Example 4

In a nitrogen atmosphere, 20 g of glycidyl methacrylate was reacted with 0.25 g of azobisisobutyronitrile in 81 g of propylene glycol monomethyl ether acetate (hereinafter, abbreviated as PGMEA) at 75° C., for 24 hours. A polyglycidyl methacrylate polymer was thus synthesized. Next, 26.30 g of a-cyano-4-hydroxycinnamic acid was graft polymerized with the epoxy groups in the polyglycidyl methacrylate polymer (20% solid in PGMEA) in the presence of 0.20 g of benzyltriethylammonium chloride. The graft polymerization reaction was performed by dissolving each of the components into a mixed solvent consisting of 464.34 g of ethyl lactate and 154.45 g of PGMEA in a mass ratio of 75:25 (ethyl lactate:PGMEA). The α-cyano-4-hydroxycinnamic acid was dissolved into the solution at about 90° C. The reaction was carried out at 120° C., for 4 to 5 hours in a nitrogen atmosphere.

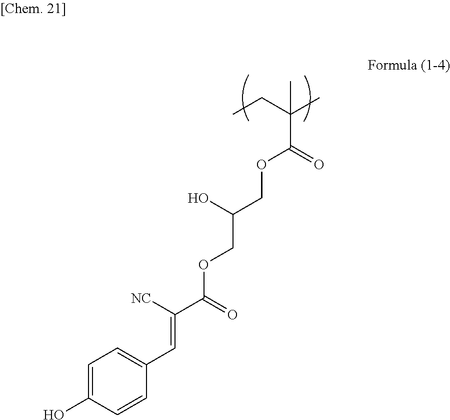

### Synthesis Example 5

A reaction flask was charged with 10.00 g of a triazinetrione-type epoxy compound (product name: TEPIC, manufactured by Nissan Chemical Corporation, epoxy functionality: 10.03 eq./kg), 12.25 g of 4-hydroxybenzaldehyde, 0.85 g of tetrabutylphosphonium bromide, and 53.90 g of propylene glycol monomethyl ether. In a nitrogen atmosphere, the materials were heated under reflux for 23 hours. Subsequently, a solution of 6.63 g of malononitrile in 15.46 g of propylene glycol monomethyl ether was added to the system. The mixture was heated under reflux for 5 hours. The reaction product obtained corresponded to Formula (1-5) and had a weight average molecular weight Mw of 800 as measured by GPC relative to polystyrene.

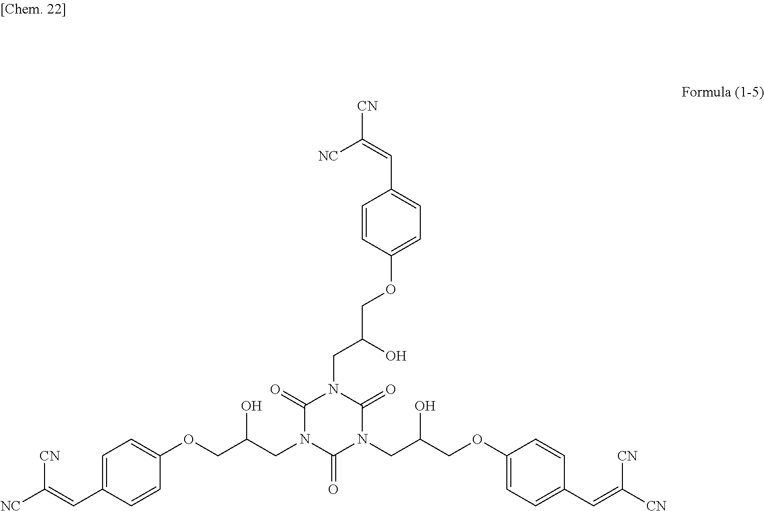 [SMILES: N#CC(C#N)=Cc1ccc(OCC(O)Cn2c(=O)n(CC(O)COc3ccc(C=C(C#N)C#N)cc3)c(=O)n(CC(O)COc3ccc(C=C(C#N)C#N)cc3)c2=O)cc1]

### Reference Synthesis Example 1

A reaction flask was charged with 3.00 g of a bisphenol A novolac-type epoxy compound (product name: jER (registered trademark) 157S70, manufactured by Mitsubishi Chemical Corporation, epoxy functionality: 4.78 eq./kg), 1.75 g of 4-hydroxybenzaldehyde, 0.12 g of tetrabutylphosphonium bromide, and 27.61 g of propylene glycol monomethyl ether. In a nitrogen atmosphere, the materials were heated at an internal temperature of 105° C., for 24 hours, while stirring. Subsequently, the reaction solution was cooled to room temperature, and a solution of 1.44 g of methyl cyanoacetate in 8.13 g of propylene glycol monomethyl ether was added to the system. The mixture was heated at an internal temperature of 105° C., for 4 hours, while stirring in a nitrogen atmosphere. The reaction product obtained corresponded to Formula (1-6) and had a weight average molecular weight Mw of 6,600 as measured by GPC relative to polystyrene.

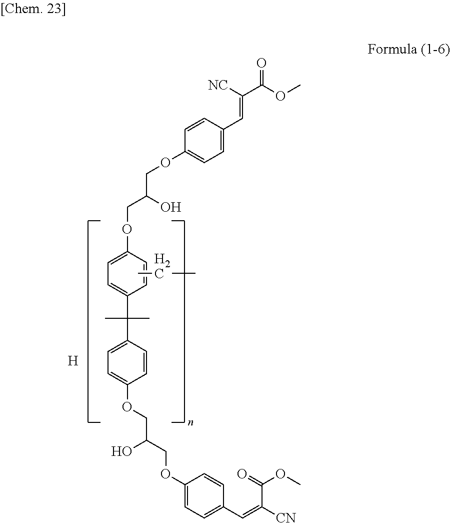

[symbol]

### Example 1

To 0.544 g of the solution of the reaction product (solid content: 17.1% by weight) corresponding to Formula (1-1) were added 0.024 g of tetramethoxymethylglycoluril (produce name: POWDER LINK [registered trademark]1174, manufactured by Cytec Industries Japan) as a crosslinking agent, 0.001 g of pyridinium-p-toluenesulfonate as a crosslinking catalyst, 0.002 g of MEGAFACE R-30N (produce name, manufactured by DIC CORPORATION), 7.18 g of propylene glycol monomethyl ether, and 1.98 g of propylene glycol monomethyl ether acetate, to prepare a solution of the resist underlayer film-forming composition.

### Example 2

To 0.568 g of the solution of the reaction product (solid content: 16.4% by weight) corresponding to Formula (1-2) were added 0.024 g of tetramethoxymethylglycoluril (produce name: POWDER LINK [registered trademark]1174, manufactured by Cytec Industries Japan) as a crosslinking agent, 0.001 g of pyridinium-p-toluenesulfonate as a crosslinking catalyst, 0.002 g of MEGAFACE R-30N (produce name, manufactured by DIC CORPORATION), 7.15 g of propylene glycol monomethyl ether, and 1.98 g of propylene glycol monomethyl ether acetate, to prepare a solution of the resist underlayer film-forming composition.

### Comparative Example 1

To 1.645 g of the solution of the reaction product (solid content: 28.6% by weight) corresponding to Formula (1-3) were added 0.118 g of tetramethoxymethylglycoluril (produce name: POWDER LINK [registered trademark]1174, manufactured by Cytec Industries Japan) as a crosslinking agent, 0.012 g of pyridinium-p-toluenesulfonate as a crosslinking catalyst, 7.521 g of propylene glycol monomethyl ether, and 0.708 g of propylene glycol monomethyl ether acetate, to prepare a solution of the resist underlayer film-forming composition.

### Comparative Example 2

To 250 g of the solution of the reaction product corresponding to Formula (1-4) were added 2.97 g of hexamethoxymethylmelamine ([produce name] CYMEL [registered trademark] 303 LF, manufactured by CYTEC) as a crosslinking agent, 0.15 g of p-toluenesulfonic acid monohydrate as a crosslinking catalyst, 91.37 g of ethyl lactate, and 30.46 g of propylene glycol monomethyl ether acetate, prepare a solution of the resist underlayer film-forming composition.

### Comparative Example 3

To 6.72 g of the solution of the reaction product (solid content: 25.8% by weight) corresponding to the Formula (1-5) were added 0.35 g of tetramethoxymethylglycoluril (produce name: POWDER LINK [registered trademark]1174, manufactured by Cytec Industries Japan) as a crosslinking agent, 0.02 g of pyridinium-p-toluenesulfonate as a crosslinking catalyst, 14.54 g of propylene glycol monomethyl ether, and 8.37 g of propylene glycol monomethyl ether acetate, to prepare a solution of the resist underlayer film-forming composition.

### Reference Example 1

To 0.847 g of the solution of the reaction product (solid content: 11.0% by weight) corresponding to the Formula (1-6) were added 0.024 g of tetramethoxymethylglycoluril (produce name: POWDER LINK [registered trademark]1174, manufactured by Cytec Industries Japan) as a crosslinking agent, 0.001 g of pyridinium-p-toluenesulfonate as a crosslinking catalyst, 0.002 g of MEGAFACE R-30N (produce name, manufactured by DIC CORPORATION), 6.87 g of propylene glycol monomethyl ether, and 1.98 g of propylene glycol monomethyl ether acetate, to prepare a solution of the resist underlayer film-forming composition.

<Evaluation of Optical Constants>

To evaluate the optical constants, each of the lithographic resist underlayer film-forming compositions prepared in Example 1 and Example 2 was applied onto a silicon wafer with a spin coater so that the film thickness would be about 50 nm, and the coatings were baked (heat treated) on a hot plate at 200° C., for 90 seconds. The resist underlayer films thus obtained were analyzed with a spectroscopic ellipsometer (VUV-VASE, manufactured by J.A. Woolam) to measure the n value (the refractive index) and the k value (the attenuation coefficient) at wavelengths of 193 nm (ArF excimer laser beam wavelength), 248 nm (KrF excimer laser beam wavelength), and 365 nm (i-radiation wavelength). Table 1 shows the results.

TABLE 1

| Example | n/k (193 nm) | n/k (248 nm) | n/k (365 nm) |
|---|---|---|---|
| Example 1 | 1.77/0.33 | 1.65/0.11 | 1.90/0.28 |
| Example 2 | 1.77/0.38 | 1.62/0.11 | 1.82/0.21 |
| Reference Example 1 | 1.63/0.49 | 1.75/0.09 | 1.82/0.21 |

In Example 1 and Example 2, the n values and the k values at 193 nm, 248 nm, and 365 nm were appropriate. From the above results, coating films from the resist underlayer film-forming compositions obtained in Example 1 and Example 2 have an anti-reflection function and are capable of reducing or eliminating the undesired factor in resist patterning, specifically, the reflection (standing waves) from the underlying substrate in the lithography process using radiations, such as ArF excimer laser beam, KrF excimer laser beam, and i-radiation. Thus, these coating films are useful as resist underlayer films.

<Evaluation of Etching Selectivity>

To evaluate the etching selectivity, each of the lithographic resist underlayer film-forming compositions prepared in Example 1, Example 2, and Comparative Example 1 to Comparative Example 3 was applied onto a silicon wafer with a spin coater so that the film thickness would be about 100 nm, and the coatings were baked (heat treated) on a hot plate at 200° C., for 90 seconds. The coating films thus obtained were dry etched with CF₄ gas using a dry etching device (product name: RIE-10NR, manufactured by Samco) to measure the ratio of the dry etching rates (the selectivity of the dry etching rates) of the resist underlayer films. Table 2 shows the results of the measurement of etching selectivity. It can be said that the larger the etching selectivity the higher the dry etching rate.

TABLE 2

| | Etching selectivity |
|---|---|
| | (relative to the etching selectivity |
| | of Comparative Example 1 taken as 1) |
| Example 1 | 1.25 |
| Example 2 | 1.34 |
| Comparative Example 1 | 1.00 |
| Comparative Example 2 | 1.16 |
| Comparative Example 3 | 1.20 |
| Reference Example 1 | 0.94 |

From the above results, it can be said that the resist underlayer films provided by the resist underlayer film compositions of Example 1 and Example 2 exhibit a larger etching selectivity and thus a higher dry etching rate than those provided by the resist underlayer film compositions of Comparative Example 1 to Comparative Example 3. That is, the former resist underlayer films enables dry etching in a shorter etching time and thus permits reduction of the loss of the resist film thickness during the removal of the resist underlayer film by dry etching. Moreover, because the shortening of the dry etching time would reduce the undesired etching damage to the substrate below the resist underlayer film, they are highly useful resist underlayer films.

<Test of Resistance to Resist Solvents>

To evaluate the resistance to resist solvents (organic solvents), each of the resist underlayer film-forming compositions prepared in Example 1 and Example 2 was applied onto a 50 nm thick copper substrate and heated at 200° C., for 90 seconds to form a resist underlayer film with a film thickness of 20 nm. Next, the copper substrate coated with the resist underlayer film composition was immersed in a common resist solvent, specifically, propylene glycol monomethyl ether acetate (PGMEA) at room temperature for 1 minute. The immersed coating film was visually inspected to evaluate the resistance. Table 3 shows the results. A coating film was judged as not having resistance to the resist solvent (organic solvent) when the coating film was removed, and judged as having resistance when it was not removed.

TABLE 3

| | Resistance of coating film |
|---|---|
| | to resist solvents (PGMEA) |
| Example 1 | Not removed |
| Example 2 | Not removed |
| Reference Example 1 | Not removed |

From the above results, the coating films from each of the resist underlayer film compositions of Example 1 and Example 2 did not removed (come off) from the copper substrate upon contact with PGMEA. Thus, it can be said that these coating films have good chemical resistance to the resist solvent. Specifically, the coating films obtained from each of the resist underlayer film compositions of Example 1 and Example 2 do not undergo any undesirable peeling phenomenon upon contact with the resist solvent and are thus useful as a resist underlayer film.

<Test of Solubility in Wet Etching Chemicals>

To evaluate the solubility in wet etching chemicals (basic organic solvents), each of the resist underlayer film-forming compositions prepared in Example 1, Example 2, and Comparative Example 1 was applied onto a 50 nm thick copper substrate and heated at 200° C., for 90 seconds to form a resist underlayer film with a film thickness of 20 nm. Next, the copper substrate coated with the resist underlayer film composition was immersed in a basic organic solvent, specifically, a 0.5% by weight dimethylsulfoxide solution of tetramethylammonium hydroxide (TMAH) at 50° C., for 5 minutes. The immersed coating film was visually inspected to evaluate the solubility. Table 4 shows the results. A coating film was judged as having good solubility (peeling property) with respect to the basic organic solvent when the coating film was removed, and was judged as not having good solubility (peeling property) when the coating film was not removed.

TABLE 4

| | Solubility of coating film |
|---|---|
| | in wet etching chemicals |
| | (0.5% by weight TMAH |
| | in dimethylsulfoxide solution) |
| Example 1 | Completely pecled |
| Example 2 | Completely peeled |
| Comparative Example 1 | Not peeled |

From the above results, the coating films formed on the copper substrate from each of the resist underlayer film compositions of Example 1 and Example 2 exhibited sufficient solubility in the wet etching chemical (basic organic solvent) as compared to the film from the resist underlayer film composition of Comparative Example 1. That is, the coating films obtained from each of the resist underlayer film compositions of Example 1 and Example 2 can exhibit good solubility (separability) in the wet etching chemical and are thus useful in the semiconductor manufacturing process that involves removing a resist underlayer film with a wet etching chemical.

### INDUSTRIAL APPLICABILITY

The resist underlayer film provided by the present invention exhibits such properties as being removable by, preferably soluble in, wet etching chemicals exclusively, while showing good resistance to resist solvents that are mainly organic solvents as well as to resist developers that are aqueous alkali solutions.

1. A resist underlayer film-forming composition comprising a solvent and a compound (A) containing a partial structure represented by Formula (1) below, 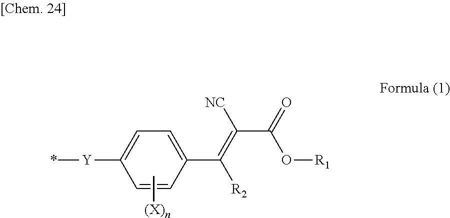
wherein R₁ and R₂ each denote a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; Y denotes a direct bond, an ether bond, a thioether bond, or an ester bond; n denotes an integer of 0 to 4; and * denotes a bond to a remaining moiety of the compound (A).

2. The resist underlayer film-forming composition according to claim 1, wherein the compound (A) is represented by Formula (2): 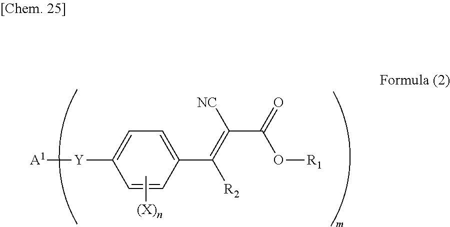
wherein A¹ denotes an m-valent organic group; m denotes an integer of 1 to 10; R₁ and R₂ each denote a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; Y denotes a direct bond, an ether bond, a thioether bond, or an ester bond; and n denotes an integer of 0 to 4.

3. The resist underlayer film-forming composition according to claim 2, wherein A¹ comprises a heterocyclic ring.

4. The resist underlayer film-forming composition according to claim 3, wherein the heterocyclic ring is triazinetrione.

5. The resist underlayer film-forming composition according to claim 2, wherein the compound (A) is a reaction product of:
a compound (a) having m quantity of epoxy group(s);
a compound (b) represented by Formula (b) below: 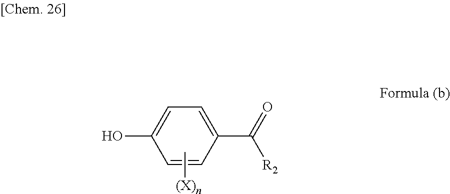
wherein R₂ denotes a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; and n denotes an integer of 0 to 4; and
a compound (c) represented by Formula (c) below: 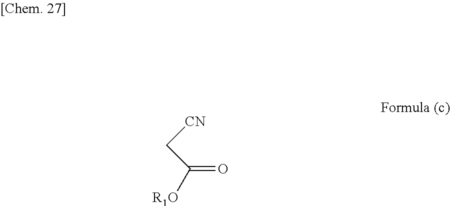
wherein R₁ denotes a hydrogen atom or an optionally substituted C1-C10 alkyl group.

6. The resist underlayer film-forming composition according to claim 1, further comprising at least one member selected from the group consisting of crosslinking agents, acids, and acid generators.

7. The resist underlayer film-forming composition according to claim 1, which is for application to a substrate having copper on a surface.

8. A resist underlayer film obtained by removing the solvent from a coating film comprising the resist underlayer film-forming composition according to claim 1.

9. A resist underlayer film comprising a dried or concentrated resist underlayer film-forming composition according to claim 1.

10. The resist underlayer film according to claim 8, which is formed on a substrate having copper on a surface.

11. A substrate comprising a copper seed layer on a surface, and the resist underlayer film of claim 8 formed on the copper seed layer.

12. A method for producing a patterned substrate, comprising the steps of:
applying the resist underlayer film-forming composition according to claim 1 onto a substrate having copper on a surface, and performing baking to form a resist underlayer film;
applying a resist onto the resist underlayer film and performing baking to form a resist film;
exposing the semiconductor substrate coated with the resist underlayer film and the resist; and
developing the exposed resist film, and performing patterning.

13. A method for manufacturing a semiconductor device, comprising the steps of:
forming, on a substrate having copper on a surface, a resist underlayer film from the resist underlayer film-forming composition according to claim 1;
forming a resist film on the resist underlayer film;
forming a resist pattern by applying a light or electron beam to the resist film followed by development, and removing the resist underlayer film exposed between the resist pattern;
plating a region exposed between the resist pattern with copper; and
removing the resist pattern and the resist underlayer film beneath the resist pattern.

14. The method according to claim 13, wherein at least one of the steps of removing the resist underlayer film is performed by wet treatment.

15. A compound (A) represented by Formula (2) below: 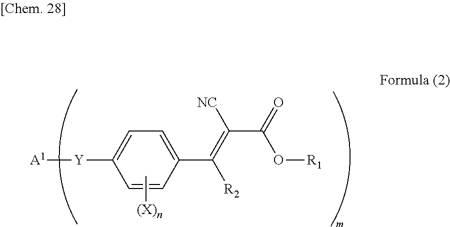
wherein A¹ denotes an m-valent organic group; m denotes an integer of 1 to 10; R₁ and R₂ each denote a hydrogen atom, a C1-C10 alkyl group, or a C6-C40 aryl group; X denotes a C1-C10 alkyl group, a hydroxy group, a C1-C10 alkoxy group, a C1-C10 alkoxycarbonyl group, a halogen atom, a cyano group, or a nitro group, or a combination thereof; Y denotes a direct bond, an ether bond, a thioether bond, or an ester bond; and n denotes an integer of 0 to 4.
